(19) United States
(12) Patent Application Publication
(10) Pub. No.: US 2009/0324656 A1
(43) Pub. Date: Dec. 31, 2009

(54) COSMETIC PREPARATIONS WITH AN ADDITIVE FROM THE BAOBAB TREE

(76) Inventor: Peter Engels, Bergisch Gladbach (DE)

Correspondence Address: KNOBBE MARTENS OLSON & BEAR LLP, 2040 MAIN STREET, FOURTEENTH FLOOR, IRVINE, CA, 92614, US

(21) Appl. No.: 12/282,640

(22) PCT Filed: Mar. 7, 2007
(86) PCT No.: PCT/EP07/01957

§ 371 Date: Feb. 17, 2009

(30) Foreign Application Priority Data
Mar. 11, 2006 (DE) 10 2006 011 314.4

(51) Int. Cl. A61K 8/97; A61K 36/00

(52) U.S. Cl. 424/401; 424/773; 424/59

(57) ABSTRACT

The present invention relates to cosmetic and dermatological preparations, in particular skincare, cosmetic and dermatological emulsions containing an additive from the baobab plant (monkey bread tree).

The present invention relates to cosmetic and dermatological preparations, in particular skincare cosmetic and dermatological emulsions with an additive from the baobab plant (monkey bread tree).

The skin is the largest human organ. Amongst its many functions (for example for temperature regulation and as a sensory organ) the barrier function, which prevents the skin (and ultimately the entire organism) from drying out, is probably the most important. At the same time, the skin acts as a protective device against the penetration and absorption of external substances. This barrier function is effected by the epidermis, which, being the outermost layer, forms the actual protective sheath against the environment. Being about one tenth of the total thickness, it is also the thinnest layer of the skin.

The epidermis is a stratified tissue in which the outer layer, the horny layer (stratum corneum), is the part which is of significance for the barrier function. The Elias skin model, which is currently recognized in the specialist field (P. M. Elias, Structure and Function of the Stratum Corneum Permeability Barrier, Drug Dev. Res. 13, 1988, 97-105) describes the horny layer as a two-component system, similar to a brick wall (bricks and mortar model). In this model, the horny cells (corneocytes) correspond to the bricks, and the lipid membrane in the intercellular spaces, which is of complex composition, corresponds to the mortar. This system is essentially a physical barrier to hydrophilic substances, but, on account of its narrow and multilayered structure, can equally, however, also only be crossed by lipophilic substances with difficulty.

Apart from its barrier action against external chemical and physical influences, the epidermal lipids also contribute to the cohesion of the horny layer and have an effect on the smoothness of the skin. In contrast to the sebaceous gland lipids, which do not form a continuous film of the skin, the epidermal lipids are distributed over the entire horny layer.

The extremely complex interaction of the moisture-binding substances and the lipids of the upper skin layers is very important for the regulation of skin moisture. Consequently, cosmetics generally comprise water-binding substances besides balanced lipid mixtures and water.

Besides the chemical composition, however, the physical behavior of these substances is also of importance. The development of very readily biocompatible emulsifiers and surfactants is therefore desirable. Products formulated therewith aid the liquid-crystalline organization of the intercellular lipids of the stratum corneum and thereby improve the barrier properties of the horny layer. It is particularly advantageous if their molecular constituents consist of substances which are naturally occurring in the epidermis.

Cosmetic skincare is primarily to be understood as meaning that the natural function of the skin as a barrier against environmental influences (e.g. dirt, chemicals, microorganisms) and against the loss of endogenous substances (e.g. water, natural fats, electrolytes) is strengthened or restored.

Impairment of this function may lead to increased absorption of toxic or allergenic substances or to attack by microorganisms and consequently to toxic or allergic skin reactions.

Another aim of skincare is to compensate for the loss by the skin of sebum and water caused by daily washing. This is particularly important when the natural regeneration ability is insufficient. Furthermore, skincare products should protect against environmental influences, in particular against sun and wind, and delay skin aging.

Medicinal topical compositions generally comprise one or more medicaments in an effective concentration. For the sake of simplicity, in order to distinguish clearly between cosmetic and medicinal use and corresponding products, reference is made to the legal provisions of the Federal Republic of Germany (e.g. Cosmetics Ordinance Foods and Drugs Act).

Customary cosmetic application forms are emulsions. These are generally understood as meaning a heterogeneous system of two liquids which are immiscible or miscible only to a limited extent with one another, which are usually referred to as phases. One is in the form of droplets (disperse or internal phase), while the other liquid forms a continuous (coherent or internal phase). Less common application forms are multiple emulsions, i.e. those which, in the droplets of the dispersed (or discontinuous) phase, comprise for their part droplets of a further dispersed phase, e.g. W/O/W emulsions and O/W/O emulsions.

If the oil phase is present in the water phase in finely distributed form, then this is an oil-in-water emulsion (O/W emulsion, e.g. milk). The basic character of an O/W emulsion is determined by the water, i.e. it is generally less greasy on the skin, is likely to be matting and absorbs more rapidly into the skin than does a W/O emulsion.

The person skilled in the art is aware of a multitude of options for formulating stable O/W preparations for cosmetic or dermatological use, for example in the form of creams and ointments, which are spreadable in the range from room temperature to skin temperature, or as lotions and milks, which are more likely flowable in this temperature range.

The stability of emulsions is dependant inter alia on their viscosity, in particular on the viscosity of the external phase. An emulsion becomes unstable when the finely dispersed particles join together again to form relatively large aggregates, and the droplets that are in contact coalesce. This process is referred to as coalescence. The more viscous the external phase of the emulsion, the slower the process of coalescence.

Emulsions of “liquid” (=flowable) consistency are used in cosmetics, for example as care lotion, cleansing lotion, face lotion or hand lotion. They generally have a viscosity of from about 2000 mPa·s to about 10 000 mPa·s. The stability of flowable emulsions is deserving of particular attention since the considerably greater mobility of the particles promotes more rapid coalescence.

Even liquid emulsions of the prior art—since they too generally comprise thickeners—are not stable towards relatively high electrolyte concentrations, which manifests itself in phase separation. It is, however, frequently desirable to use certain electrolytes, such as, for example, water-soluble UV filters, in order to be able to utilize their other physical, chemical and physiological properties. Although in many cases appropriate choice of the emulsifier system can provide remedies to a certain extent, other disadvantages then arise just as often.

The disadvantages mentioned can, for example, lie in the fact that emulsifiers, like ultimately any chemical substance, can in individual cases trigger allergic reactions or reactions based on oversensitivity of the user, although the use of customary cosmetic emulsifiers is generally completely risk-free.

In order to be able to ensure the metastability of emulsions, interface-active substances, i.e. emulsifiers, are usually necessary. The use per se of the customary cosmetic emulsifiers is completely risk-free. Nevertheless, emulsifiers, like ultimately any chemical substance, can in individual cases trigger allergic reactions or reactions based on oversensitivity of the user. For example, it is known that in some particularly sensitive people, certain photodermatoses are triggered by certain emulsifiers and simultaneous action of sunlight.

It is possible to produce emulsifier-free preparations which, for example, have water droplets dispersed in an oil phase, or oil droplets dispersed in a water phase. Such systems are sometimes called oleodispersions or hydrodispersions depending on which is the disperse phase and which is the continuous phase.

For cosmetics technology, however, it is neither necessary nor possible to dispense with emulsifiers entirely, especially since there is a certain choice of particularly mild emulsifiers. However, the prior art lacks a satisfactorily broad range of such emulsifiers which would then also significantly broaden the application spectrum of correspondingly mild and skin-compatible cosmetic preparations.

The harmful effect of the ultraviolet part of solar radiation on the skin is generally known. While rays with a wavelength of less than 290 nm (the so-called UVC region) are absorbed by the ozone layer in the earth's atmosphere, rays in the range between 290 nm and 320 nm, the so-called UVB region, cause erythema, simple sunburn or even burns of varying severity.

The erythema activity maximum of sunlight is given as the relatively narrow region around 308 nm.

Numerous compounds are known for protecting against UVB radiation; these are derivatives of 3-benzylidenecamphor, of 4-aminobenzoic acid, of cinnamic acid, of salicylic acid, of benzophenone, of 2-phenyl-benzimidazole, of acrylic acid, of cinnamic acid, of aminobenzoic acid and of triazine, and also silicone derivatives.

It is also important to have available filter substances for the range between about 320 nm and about 400 nm, the so-called UVA region, since its rays too can cause damage. For a long time it has been incorrectly assumed that the long-wave UV-A radiation with a wavelength between 320 nm and 400 nm has only a negligible biological effect and that, accordingly, the UV-B rays are responsible for most photodamage to the human skin. In the meantime, however, it has been demonstrated by numerous studies that UV-A radiation is much more harmful than UV-B radiation with regard to the triggering of photodynamic, specifically phototoxic, reactions and chronic changes in the skin. Also, the harmful effect of UV-B radiation can be further intensified by UV-A radiation.

For example, it has inter alia been proven that even the UV-A radiation under very normal everyday conditions is enough to damage, within a short period, collagen and elastin fibers, which are of essential importance for the structure and strength of the skin. This leads to chronic photoinduced skin changes—the skin “ages” prematurely. The clinical appearance of skin aged by light includes, for example, wrinkles and lines, and an irregular, furrowed relief. In addition, the parts affected by photoinduced skin aging can have irregular pigmentation. The formation of brown spots, keratoses and even carcinomas or malignant melanomas is also possible. Skin aged prematurely as a result of everyday UV exposure is characterized, moreover, by lower activity of the Langerhans cells and slight, chronic inflammation.

Approximately 90% of the ultraviolet radiation reaching the earth consists of UV-A rays. While the UV-B radiation varies widely depending on numerous factors (e.g. time of year and day or degree of latitude), the UV-A radiation remains relatively constant day after day irrespective of the time of year and day or geographical factors. At the same time, the majority of UV-A radiation penetrates into the living epidermis, whereas about 70% of UV-B rays are retained by the horny layer.

Preventive protection against UV-A rays, for example by applying photoprotective filter substances to the skin in the form of a cosmetic or dermatological formulation, is consequently of fundamental importance.

Generally speaking, the photoabsorption behavior of photoprotective filter substances is very well known and documented, not least because most industrialized countries have positive lists for the use of such substances, which impose very strict standards on the documentation. For the concentration of the substances in the finished formulations, the absorbance values can at best be a guide, since interaction with substances within the skin or surface of the skin itself may result in imponderables. In addition, it is usually difficult to estimate beforehand how uniformly and thickly the filter substance is distributed in and on the horny layer of the skin. To test the UV-A protection performance, use is usually made of the IPD method (IPD [identical to] immediate pigment darkening). Similarly to the determination of the sun protection factor, this method gives a value which indicates how much longer the skin protected with the photoprotective composition can be irradiated with UV-A radiation before the pigmentation which occurs is the same as for unprotected skin.

Another test method which has become established throughout Europe is the Australian standard AS/NZS 2604: 1997. Here, the absorption of the preparation in the UV-A region is measured. In order to meet the standard, the preparation must absorb at least 90% of the UV-A radiation in the range 320-360 nm.

The use concentration of known photoprotective filter substances which, in particular, also exhibit a high filter effect in the UV-A region, is often limited by the very fact that they are combined with other substances which are in the form of solids. There are therefore certain formulation difficulties in achieving relatively high sun protection factors and UV-A protection performance.

However, UV radiation can also lead to photochemical reactions, where the photochemical reaction products interfere with the skin's metabolism. Such photochemical reaction products are predominantly free-radical compounds, for example hydroxy radicals. Undefined free-radical photo products which form in the skin itself can also exhibit uncontrolled secondary reactions on account of their high reactivity. However, singlet oxygen, a non-free-radical excited state of the oxygen molecule, can also arise during UV irradiation, as can short-lived epoxides and many others. Singlet oxygen, for example, is distinguished from normal triplet oxygen (free-radical ground state) by virtue of its increased reactivity. However, excited, reactive (free-radical) triplet states of the oxygen molecule also exist.

In order to prevent these reactions, it is known to additionally incorporate antioxidants and/or free-radical scavengers into cosmetic and dermatological formulations.

The compounds which are used as photoprotective agents for cosmetic and dermatological photoprotective formulations are mostly characterized per se by good photoprotective effect. However, they have the disadvantage that it is sometimes difficult to incorporate them into such formulations in a satisfactory manner.

The sun protection factor (SPF) indicates how much longer the skin protected with the photoprotective composition can be irradiated until the same erythema reaction occurs as for unprotected skin (i.e. ten times as long compared with unprotected skin in the case of SPF=10).

In any case, the consumer expects, on the one hand, reliable information from the manufacturer regarding the sun protection factor, not least because of the discussion about the “hole in the ozone layer” which has become a topic of public interest, and on the other hand there is a tendency by the consumer toward relatively high and high sun protection factors.

Since photoprotective filter substances are generally expensive and since some photoprotective filter substances are additionally difficult to incorporate into cosmetic or dermatological preparations in relatively high concentrations, it was an object of the invention to arrive at, in a simple and cost-effective manner, preparations which, even having unusually low concentrations of photoprotective filter substances, nevertheless achieve acceptable or even high SPF values.

It was a further object of the invention to conceive cosmetic or dermatological photoprotective preparations which have a high stability of the photoprotective filters.

It was a further object of the present invention to conceive cosmetic or dermatological photoprotective preparations which are characterized by an increased care effect.

It was a further object of the present invention to provide cosmetic and dermatological preparations into which natural products are incorporated.

The use of different constituents of the baobab plant as foods and for various other purposes is generally known. Thus, for example, the leaves are freshly eaten as vegetables, or used in dried and ground form for the preparation of foods, such as sauces, broths, etc. The fruit pulp of the baobab plant is, on account of its vitamin content, either eaten fresh, added to cooked dishes or, on account of its pleasant taste, used as a base in the manufacture of beverages. The seeds of the baobab plant are used as binders for soups, roasted as snacks or as a substitute base for the production of coffee, pressed as cooking oil base etc. The ground roots of the baobab plant are used as a base for the manufacture of dyes. In medicine, the bark of the baobab plant with its astringent, diaphoretic and even antipyretic properties is used, the fruit flesh and the seeds are used in cases of fever or on inflamed wounds on account of the anti-inflammatory properties. By contrast, the leaves are used to combat sweating, kidney and bladder complaints and as an antiasthmatic agent. Cosmetic applications of various extracts from individual constituents of the baobab are likewise known.

EP 0 973 494 describes, for example, the use of an extract from leaves of a plant of the genus Adansonia, in particular of the species Adansonia digitata (monkey bread tree=baobab), for cosmetic, dermatological and pharmaceutical applications, and also a cosmetics and/or medicament product or a cosmetics or medicament composition for the skin and/or the epithelia appendages with such an extract.

The Korean patent application with the publication number 1020040015586 A describes a cosmetic composition comprising a baobab extract which is stabilized with nanoliposomes.

It was an aim of the present invention to provide cosmetic and dermatological preparations, in particular skincare cosmetic and dermatological emulsions with an additive from the baobab plant which offer particularly effective skin protection and skincare.

It was a further aim of the present invention to provide cosmetic and dermatological preparations, in particular skincare cosmetic and dermatological emulsions, which have a high stability of the UV filters incorporated into the preparations.

These objects are achieved by a cosmetic and/or dermatological preparation comprising an additive of at least one constituent of a plant of the genus Adansonia, in particular of the species Adansonia digitata (baobab plant), A. grandidieri, A. za, or A. gibbosa, where this additive is not obtained by hot extraction.

According to the present invention, an additive from at least one constituent of the baobab plant in which the constituents naturally occurring in the baobab plant are present without significant degeneration is incorporated into a cosmetic or dermatological “base composition”, which can comprise customary constituents of such a particular composition, without any intention to thereby limit the invention. This is intended to mean that such additives are produced from the plant part(s) without the substances located in the plant parts being subjected to high, e.g. thermal, stress. The plant constituents can, if appropriate, be depectinated, i.e. subjected to a treatment which reduces the pectin content of the constituent/degrades the pectin.

Hitherto, essentially extracts from plants (parts) which have been produced beforehand from the plant parts with strong heating have primarily been incorporated into cosmetics. For example, the aforementioned EP-A 0 973 494 describes the production of an extract from baobab leaves, during which the stock is heated for a relatively long time at up to 95° C. before the solid constituents are removed.

According to the present invention, the constituents of the plant are incorporated into the cosmetic or dermatological compositions either directly in fresh or dried, finely comminuted form, or an aqueous or organic solvent extract is produced from the constituents which has only been heated to at most moderate temperatures during production. Moderate temperatures are to be understood here as meaning a temperature of at most 65° C., preferably of at most 50° C., particularly preferably of at most 40° C.

Drying of the constituent/constituents can take place by one of the known types of drying, with either air drying or freeze-drying being preferred. A temperature increase during drying is possible, but the temperature should preferably not exceed the aforementioned values here either. Drying under environmental conditions in the countries of manufacture, thus e.g. Africa with daytime temperatures sometimes in the region of more than 40° C. (in the sun also significantly higher), however, likewise falls within the scope of the present invention. Provided the plant constituents are not subjected to an extraction method, the temperature is not as disadvantageous for the substances located in the plant parts as after leaching out from the “natural environment” by an extraction method. Consequently, in the case of the direct incorporation of the plant constituents (without extraction method) into the preparations described here, the temperature applied during drying is not limiting for the invention.

By virtue of this type of work-up of the plant constituents, thus either (drying and) comminution or with (if appropriate additional) use of a temperature-limited extraction step, the substances present in the plant constituents are treated in a particularly gentle manner. As a result, the additives from constituents of the baobab plant incorporated into the preparations according to the invention have a largely “original” composition, i.e. a composition as also exists in the used constituent(s) in their natural environment.

Many of the substances found in the constituents of the plant are exceptionally labile toward the effect of heat—especially outside of the “protective” plant structure. Consequently, these are often largely destroyed in an extraction method in which strong heating takes place. Therefore, in one embodiment according to the invention, an extract from at least one constituent of the baobab plant is produced in which the constituent(s) in an extractant are subjected to a temperature above 65° C. for a maximum of a few seconds (at most 10). Preferably, a temperature of more than 65° C. is not reached. In one embodiment of the invention, such a “cold” extract is then incorporated into a cosmetic or dermatological preparation. Suitable extractants for such an extraction are, for example, water (also with the addition of various soluble substances such as, for example, salts or buffer substances), an organic solvent such as methanol, ethanol, butanol, propanol, isopropanol, acetone, chloroform or the like or an oil, in particular one of those specified below for the formulations, without, however, being limited thereto.

In a further embodiment, the plant constituents are not subjected to an extraction method, but are either comminuted in the fresh or dried state, the resulting particles are, if appropriate, separated according to size and incorporated directly into a cosmetic or dermatological composition. The plant constituents can be comminuted by any suitable method, such as, for example, titration, grinding, cutting, chopping or the like, without being limited thereto. After the comminution, the prepared powder or mush can be separated into size fractions by suitable measures, e.g. by sieving (in particular in the case of powders), slurrying (in the case of mush) etc., so that the particles incorporated into the compositions have, if desired, a certain size distribution. However, this is not obligatory for the invention, but instead depends on the processing parameters of the composition to be produced.

According to the present invention, constituents of a plant of the genus Adansonia, in particular the species Adansonia digitata (monkey bread tree=baobab), A. grandidieri, A. za, or A. gibbossa directly or extracts therefrom which have been produced with at most moderate heating, can be incorporated into cosmetic or dermatological compositions. Suitable constituents of the plants are, in particular, the fruit pulp, the leaves, the bark, the seeds contained in the fruit pulp, the flowers and the roots. Each of these constituents can be used individually according to the invention, or else a mixture of two or more of these constituents.

The various constituents of the plants comprise mixtures of different substances which are particularly suitable for use in cosmetic and dermatological composition. Thus, the leaves comprise primarily many proteins, a high calcium content and a high content of vitamins A and E, the fruit pulp is characterized by a high vitamin C and calcium content, and also by a high fraction of pectin, the seeds comprise oils with unsaturated fatty acids and have a high protein content. Since these are natural products, a specific concentration of a substance or a ratio of the substances to one another cannot be determined definitively, although reference is made to the following references: Gebauer J. et al. Gartenbauwissenschaft (2002), 67 (4), pp. 155-160; Nour, A. et al., Trop. Sci (1980), 22 (4), pp. 383-388.

In the present application, wounded or inflamed skin is understood as meaning skin that is irritated and reddened on account of high mechanical, chemical or thermal stress. Examples thereof are sunburn, wounds, skin scratched after itching, and the like.

For the purposes of the invention, mature skin is understood as meaning intrinsically and extrinsically aged skin which is clinically characterized inter alia by the appearance of wrinkles, by increased dryness and increased skin roughness. The cause of mature skin is dermal and epidermal changes which start firstly with increasing age, secondly are accelerated by external influences.

Within the context of the invention, age-dry skin means an increase in dry skin conditions with increasing age, which requires an increasing care requirement. Balancing out degenerative processes in all skin layers within the context of the invention means a normalization and improvement in those processes in the skin which are disrupted, for example, depending on age and UV.

Improvement in the communication between the individual skin layers (dermal-epidermal crosstalk) within the context of the invention means that the exchange of cellular messengers between dermis, epidermis and basal membrane is improved such that cell functions are beneficially influenced.

Treatment and balancing out of the glucosaminoglycan/proteoglycan metabolism within the context of the invention means that the age- or UV-induced changes are counteracted with the help of the active ingredients described here.

Treatment and balancing out of moisture deficiencies in the upper and lower skin layers (epidermal and dermal) within the context of the invention means that through topical treatment with the cosmetic and dermatological preparations described here, it is possible to normalize and/or to improve the hydration of the skin, which is essential for optimum cellular and noncellular functions. Within the context of the invention, structural improvement in the skin is understood as meaning an improvement in the essential structure-imparting fibers of the skin, i.e. primarily collagen and elastin fibers.

Within the context of the invention, strengthening the epidermal-dermal junction zone means an improvement in the dermal-epidermal intermeshing, with the counteraction of age-related processes.

Increasing the anchor fibrils (collagen-7) and the collagen synthesis within the context of the invention means an increase in the collagen synthesis in the skin.

It can be established that the preparations according to the invention
have a very good moisturizing effect,
promote skin smoothing,
are characterized by a high care effect,
are characterized by very good biocompatibility,
are characterized by a good skin feel,
bring about good evening out of moisture deficiencies in upper and lower skin layers. (epidermal and dermal),
contribute to structural improvement of the skin, particularly in old age,
can be used for better care and prophylaxis against skin exposed to sun (sun-burnt skin, photoaged skin and/or age-dry skin),
are suitable for balancing out degenerative processes in all skin layers,
contribute to the improvement in communication between the individual skin layers (dermal-epidermal crosstalk),
contribute to the increase in the anchor fibrils (collagen-7) and in collagen synthesis and
have broad cosmetic variability and can be formulated over broad consistency and viscosity ranges from 400 mPas to >20 000 mPas.

It is preferred that the preparation according to the invention is in the form of an O/W emulsion or hydrodispersion.

Basic constituents (in the “base formulation”) that can be used for the preparations according to the invention are:
water or aqueous solutions
aqueous ethanolic solutions
natural oils and/or chemically modified natural oils and/or synthetic oils;
fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;
alcohols, diols or polyols of low carbon number, and ethers thereof, preferably ethanol, isopropanol, propylene glycol, glycerol, octoxyglycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents can be used.

Within the context of the present disclosure, the expression “lipids” is sometimes used as a genetic term for fats, oils, waxes and the like, said expression being entirely commonplace to the person skilled in the art. The terms “oil phase” and “lipid phase” are also used synonymously.

Oils and fats differ inter alia in their polarity, which is difficult to define. It has already been proposed to adopt the interfacial tension toward water as a measure of the polarity index of an oil or of an oil phase. Then, the lower the interfacial tension between this oil phase and water, the greater the polarity of the oil phase in question. According to the invention, the interfacial tension is regarded as one possible measure of the polarity of a given oil component.

The interfacial tension is the force which acts on an imaginary line one meter in length in the interface between two phases. The physical unit for this interfacial tension is conventionally calculated from the force/length relationship and is usually expressed in mN/m (millinewtons divided by meters). It has a positive sign if it endeavors to make the interface smaller. In the converse case, it has a negative sign. Within the context of the present invention, lipids are regarded as polar if their interfacial tension toward water is less than 20 mN/m, and as nonpolar if their interfacial tension toward water is more than 30 mN/m. Lipids with an interfacial tension toward water between 20 and 30 mN/m are generally referred to as medium-polarity.

Polar oils are for example those from the group of lecithins and the fatty acid triglycerides, namely the triglycerol esters of saturated and/or unsaturated, branched and/or unbranched alkane carboxylic acids of chain length from 8 to 24, in particular 12 to 18, carbon atoms. The fatty acid triglycerides can, for example, be advantageously selected from the group of synthetic, semisynthetic and natural oils, such as, for example, olive oil, sunflower oil, soya oil, peanut oil, rapeseed oil, almond oil, palm oil, coconut oil, castor oil, wheatgerm oil, grapeseed oil, safflower oil, evening primrose oil, macadamia nut oil, baobab oil, avocado oil and the like.

Further polar oil components can be selected from the group of esters of saturated and/or unsaturated, branched and/or unbranched alkane carboxylic acids of chain length from 3 to 30 carbon atoms and saturated and/or unsaturated, branched and/or unbranched alcohols of chain length from 3 to 30 carbon atoms, and also from the group of esters of aromatic carboxylic acids and saturated and/or unsaturated, branched and/or unbranched alcohols of chain length from 3 to 30 carbon atoms. Such ester oils can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, such as, for example, jojoba oil.

In addition, the oil phase can advantageously be selected from the group of dialkyl ethers, the group of saturated or unsaturated, branched or unbranched alcohols. It is particularly advantageous if the oil phase of the W/O emulsions according to the invention has a content of C₁₂₋₁₅-alkyl benzoate or consists entirely of this.

In addition, the oil phase can advantageously be selected from the group of Guerbet alcohols. Guerbet alcohols are named after Marcel Guerbet who described their preparation for the first time. They are formed according to the reaction equation

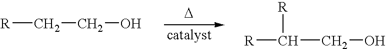

by oxidation of an alcohol to an aldehyde, by aldol condensation of the aldehyde, elimination of water from the aldol and hydrogenation of the allyl aldehyde. Guerbet alcohols are liquid even at low temperatures and cause virtually no skin irritations. They can be used advantageously as fatting, superfatting and also refatting constituents in skincare and haircare compositions.

The use of Guerbet alcohols in cosmetics is known per se. Such species are then in most cases characterized by the structure

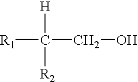

Here, R₁ and R₂ are usually unbranched alkyl radicals.

According to the invention, the Guerbet alcohol or alcohols is/are advantageously selected from the group in which

R₁=propyl, butyl, pentyl, hexyl, heptyl or octyl and

R₂=hexyl, heptyl, octyl, nonyl, dedyl, undecyl, dodecyl, tridecyl or tetradecyl.

Guerbet alcohols preferred according to the invention are 2-butyloctanol, which is available, for example, under the trade name Isofol® 12 from Condea Chemie GmbH, and. 2-hexyldecanol, which is available, for example, under the trade name Isofol® 16 from Condea Chemie GmbH.

Mixtures of Guerbet alcohols according to the invention can also be used advantageously according to the invention. Mixtures of 2-butyloctanol and 2-hexyl-decanol are obtainable, for example, under the trade name Isofol® 14 from Condea Chemie GmbH.

The total amount of Guerbet alcohols in the finished cosmetic or dermatological preparations is advantageously selected from the range up to 25.0% by weight, preferably 0.5-15.0% by weight,. based on the total weight of the preparations.

Any desired mixtures of such oil and wax components are also to be used advantageously within the context of the present invention. It may also in some cases be advantageous to use waxes, for example cetyl palmitate, as the sole lipid component of the oil phase.

Nonpolar oils are, for example, those which are selected from the group of branched and unbranched hydrocarbons and hydrocarbon waxes, in particular Vaseline (petrolatum), paraffin oil, squalane and squalene, polyolefins and hydrogenated polyisobutenes. Among the polyolefins, polydecenes are the preferred substances. Table 1 below lists lipids which are advantageous according to the invention as individual substances or else in a mixture with one another. The corresponding interfacial tensions toward water are given in the last column. However, it is also advantageous to use mixtures of greater or lesser polarity and the like.

TABLE 1

| Trade name | INCI name | (mN/m) |
|---|---|---|
| Isofol ® 14 T | Butyl decanol + hexyl | 27.6 |
| | decanol + hexyl octanol + butyl | |
| | octanol | |
| Isofol ® 16 | Hexyldecanol | 24.3 |
| Eufanol ® G | Octyldodecanol | 24.8 |
| Cetiol ® OE | Dicaprylyl ether | 22.1 |
| Miglyol ® 812 | Caprylic/capric triglyceride | 21.3 |
| Cegesoft ® C24 | Octyl palmitate | 23.1 |
| Isopropyl stearate | Isopropyl stearate | 21.9 |
| Estol ® 1540 EHC | Octyl octanoate | 30.0 |
| Finsolv ® TN | C₁₂₋₁₅ alkyl benzoate | 21.8 |
| Cetiol ® SN | Cetearyl isononanoate | 28.6 |
| Demofeel ® BGC | Butylene glycol | 21.5 |
| | dicaprylate/dicaprate | |
| Trivent ® OCG | Tricaprylin | 20.2 |
| MOD | Octyldodeceyl myristate | 22.1 |
| Cosmacol ® ETI | Di-C₁₂₋₁₃ alkyl tartrate | 29.4 |
| Miglyol ® 829 | Caprylic/capric diglyceryl | 29.5 |
| | succinate | |
| Prisorine ® 2036 | Octyl isostearate | 29.7 |
| Tegosoft ® SH | Stearyl heptanoate | 29.7 |
| Abil ® Wax 9840 | Cetyl dimethicone | 25.1 |
| Cetiol ® LC | Coco caprylate/caprate | 24.8 |
| IPP | Isopropyl palmitate | 22.5 |
| Luvitol ® EHO | Cetearyl octanoate | 28.6 |
| Cetiol ® 868 | Octyl stearate | 28.4 |

Within the context of the present invention, particularly advantageous polar lipids are all native lipids, such as, for example, olive oil, sunflower oil, soya oil, peanut oil, rapeseed oil, almond oil, palm oil, coconut oil, castor oil, wheatgerm oil, grapeseed oil, safflower oil, evening primrose oil, macadamia nut oil, corn oil, avocado oil, oil from the cores of the baobab fruit and the like, and those listed below.

| Trade name | INCI name | Polarity (mN/m) |
|---|---|---|
| Isofol ® 14 T | Butyl decanol (+) hexyl | 19.8 |
| | decanol (+) hexyl octanol | |
| | (+) butyl octanol | |
| Lipovol MOS-130 | Tridecyl stearate (+) | 19.4 |
| | tridecyl trimellitate (+) | |
| | dipentaerythrityl | |
| | hexacaprylate/hexacaprate | |
| Castor oil | | 19.2 |
| Isofol Ester 0604 | | 19.1 |
| Miglyol 840 | Propylene glycol | 18.7 |
| | dicaprylate/dicaprate | |
| Isofol 12 | Butyl octanol | 17.4 |
| Tegosoft SH | Stearyl heptanoate | 17.8 |
| Avocado oil | | 14.5 |
| Cetiol B | Dibutyl adipate | 14.3 |
| Dermol 488 | PEG 2 diethylene hexanoate | 10.1 |
| Cosmacol ELI | C₁₂₋₁₃ alkyl lactate | 8.8 |
| Dermol 489 | Diethylene glycol | 8.6 |
| | dioctanoate/diisononanoate | |
| Cosmacol ETI | Di-C12/13 alkyl tartrate | 7.1 |
| Emerest 2384 | Propylene glycol | 6.2 |
| | monoisostearate | |
| Myritol 331 | Cocoglycerides | 5.1 |
| Prisorine 2041 GTIS | Triisostearin | 2.4 |

Particularly advantageous medium-polarity lipids within the context of the present invention are the substances listed below:

| Trade name | INCI name | Polarity (mN/m) |
|---|---|---|
| DUB VCI 10 | Isodecyl neopentanoate | 29.9 |
| Dermol IHD | Isohexyl decanoate | 29.7 |
| Dermol 108 | Isodecyl octanoate | 29.6 |
| Dihexyl ether | Dihexyl ether | 29.2 |
| Dermol 109 | Isodecyl 3,5,5 trimethyl | 29.1 |
| | hexanoate | |
| Cetiol SN | Cetearyl isononanoate | 28.6 |
| Isopropyl palmitate | Isopropyl palmitate | 28.8 |
| Jojoba oil gold | | 26.2 |
| Wacker AK 100 | Dimethicone | 26.9 |
| Dermol 98 | 3,5,5-Trimethyl 2-ethyl- | 26.2 |
| | hexanoate | |
| Eutanol G | Octyldodecanol | 24.8 |
| Isofol 16 | Hexyl decanol | 24.3 |
| Dermol 139 | Isotridecyl 3,5,5- | 24.5 |
| | trimethylhexanonanoate | |
| Cetiol PGL | Hexyldecanol (+) hexyl dexyl | 24.3 |
| | laurate | |
| Cegesoft C24 | Octyl palmitate | 23.1 |
| M.O.D. | Octyldodeceyl myristate | 22.1 |
| Macadamia nut oil | | 22.1 |
| Silicone oil VP | Phenyl trimethicone | 22.7 |
| 1120 | | |
| Isocarb 12 | Butyl octanoic acid | 22.1 |
| Isopropyl stearate | Isopropyl stearate | 21.9 |
| Finsolv TN | C₁₂₋₁₅ alkyl benzoate | 21.8 |
| Dermofeel BGC | Butylene glycol | 21.5 |
| | caprylate/caprate | |
| Miglyol 812 | Caprylic/capric triglyceride | 21.3 |
| Trivent OCG | Tricaprylin | 20.2 |
| Dermol 866 | PEG diethylhexanoate-/ | 20.1 |
| | diisononoate/ethylhexyl | |
| | isononanoate | |

Particularly advantageous nonpolar lipids. within the context of the present invention are the substances listed below:

| Trade name | INCI name | Polarity (mN/m) |
|---|---|---|
| Ecolane 130 | Cycloparaffin | 49.1 |
| Nexbase 2006 FG | Polydecene | 46.7 |
| Polysylane | Hydrogenated polyisobutene | 44.7 |
| Wacker Silicone oil | Polydimethylsiloxane | 46.5 |
| AK 50 | | |
| Solvent ICH | Isohexadecane | 43.8 |
| Pionier 2076 | Mineral oil | 43.7 |
| Pionier 6301 | Mineral oil | 43.7 |
| Wacker silicone oil | Polydimethylsiloxane | 42.4 |
| AK 35 | | |
| Isoeicosane | Isoeicosane | 43.9 |
| Isofol 1212 | | 40.3 |
| Carbonate | | |
| Softcutol O | Ethoxydiglycol oleate | 40.5 |
| Lipodermanol OL | Decyl olivate | 40.3 |
| Cetiol S | Dioctylcyclohexane | 39.0 |
| Pionier 2071 | Mineral oil | 38.3 |
| Hydrobrite 1000 PO | Paraffinum liquidum | 37.6 |
| Tegosoft HP | Isocetyl palmitate | 36.2 |
| Isofol ester 1693 | | 33.5 |
| Isofol ester 1260 | | 33.0 |
| Prisorine 2036 | Octyl isostearate | 31.6 |
| Cetiol CC | Dicaprylyl carbonate | 31.7 |
| Dermol 99 | Trimethylhexyl isononanoate | 31.1 |
| Dermol 89 | 2-Ethylhexyl isononanoate | 31.0 |
| Cetiol OE | Dicaprylyl ether | 30.9 |
| Dihexyl carbonate | Dihexyl carbonate | 30.9 |
| Silkflo 366 NF | Polydecene | 30.1 |
| Estol 1540 EHC | Octyl cocoate | 30.0 |

However, it is also advantageous to use mixtures of higher and lower polarity lipids and the like. Thus, the oil phase can advantageously be selected from the group of branched and unbranched hydrocarbons and hydrocarbon waxes, dialkyl ethers, the group of saturated or unsaturated, branched or unbranched alcohols, and fatty acid triglycerides, namely the triglycerol esters of saturated and/or unsaturated, branched and/or unbranched alkane carboxylic acids of chain length from 8 to 24, in particular 12-18, carbon atoms. The fatty acid diglycerides can, for example, be advantageously selected from the group of synthetic, semisynthetic and natural oils, for example olive oil, sunflower oil, soya oil, peanut oil, rapeseed oil, almond oil, palm oil, coconut oil, palm kernel oil and the like.

It may likewise be advantageous to select the oil phase of the preparations according to the invention partially or completely from the group of cyclic and/or linear silicones, which are also referred to within the context of the present disclosure as “silicone oils”. Such silicones or silicone oils may be present as monomers, which are generally characterized by structural elements as follows:

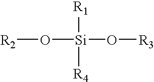

Linear silicones having two or more siloxyl units which are to be used advantageously according to the invention are generally characterized by structural elements as follows:

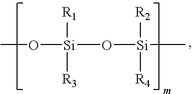

where the silicon atoms can be substituted by identical or different alkyl radicals and/or aryl radicals, which are represented here in general terms by the radicals R₁-R₄ (it should be said that the number of different radicals is not necessarily limited to 4). m can assume values from 2-200 000.

Systematically, the linear silicone oils are referred to as polyorganosiloxanes; the methyl-substituted polyorganosiloxanes, which constitute the compounds of this group that are most important in terms of amount and are characterized by the following structural formula

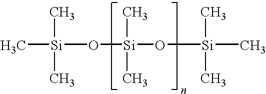

are also referred to as polydimethylsiloxane or dimethicone (INCI). Dimethicones come in a variety of chain lengths and with various molecular weights. Dimethicones of varying chain length and phenyltrimethicones are particularly advantageous linear silicone oils within the context of the present invention.

Particularly advantageous polyorganosiloxanes within the context of the present invention are also, for example, dimethylpolysiloxanes [poly(dimethyl-siloxane)], which are commercially available, for example, under the trade names ABIL 10 to 10 000. Also advantageous are phenylmethylpolysiloxanes (INCI: Phenyl Dimethicone, Phenyl Trimethicone), amino-modified silicones (INCI: Amodimethicone) and silicone waxes, e.g. polysiloxane-polyalkylene copolymers (INCI: Stearyl Dimethicone and Cetyl Dimethicone) and dialkoxydimethylpolysiloxanes (stearoxy dimethicone and behenoxy stearyl dimethicone), which are available as various abil wax grades.

The aqueous phase of the emulsion-based preparations advantageously comprises, if appropriate, alcohols, diols or polyols of low carbon number, and ethers thereof, preferably ethanol, isopropanol, propylene glycol, glycerol, ethylene glycol, ethylene glycol mononethyl or monobutyl ethers, propylene glycol monomethyl, monoethyl or monobutyl ethers, diethylene glycol monomethyl or monoethyl ethers and analogous products, also alcohols of low carbon number, e.g. ethanol, isopropanol, 1,2-propanediol, glycerol.

One advantage of the present invention is that it permits the use of high concentrations of polyols, in particular glycerol.

Preferably, emulsions according to the present invention can comprise one or more hydrocolloids.

Cyclic silicones to be used advantageously according to the invention are generally characterized by structural elements as follows

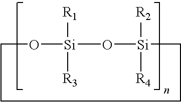

where the silicon atoms can be substituted by identical or different alkyl radicals and/or aryl radicals, which are represented here in general terms by the radicals R1-R4 (it should be said that the number of different radicals is not necessarily limited to 4). n can assume values of 3/2 to 20. Fractional values for n take into consideration that uneven numbers of siloxyl groups may be present in the cycle.

Phenyltrimethicone is advantageously selected as silicone oil. Other silicone oils, for example dimethicone, phenyldimethicone, cyclomethicone (octa-methylcyclotetrasiloxane), for example hexamethyl-cyclotrisiloxane, polydimethylsiloxane, poly(methyl-phenylsiloxane), cetyldimethicone, behenoxydimethicone can also be used advantageously within the context of the present invention.

Mixtures of cyclomethicone. and isotridecyl isononanoate, and also those of cyclomethicone and 2-ethylhexyl isostearate are also advantageous.

However, it is also advantageous to select silicone oils of similar constitution to the compounds referred to above whose organic side. chains are derivatized, for example polyethoxylated and/or polypropoxylated. These include, for example, polysiloxane-polyalkyl-polyether copolymers, such as cetyldimethicone copolyol, (cetyldimethicone copolyol (and) polyglceryl-4 isostearate (and) hexyl laurate). The oil phase can also advantageously be selected from the group of branched and unbranched hydrocarbons and hydrocarbon waxes, dialkyl ethers, the group of saturated or unsaturated, branched or unbranched alcohols, and fatty acid triglycerides, namely the triglycerol esters of saturated and/or unsaturated, branched and/or unbranched alkane carboxylic acids of chain length from 8 to 24, in particular 12-18 carbon atoms. The fatty acid triglycerides can, for example, be advantageously selected from the group of synthetic, semisynthetic and natural oils, e.g. olive oil, sunflower oil, soya oil, peanut oil, rapeseed oil, almond oil, palm oil, coconut oil, palm kernel oil and the like.

Suitable fat and/or wax components to be used can be selected from the group of vegetable waxes, animal waxes, mineral waxes and petrochemical waxes. According to the invention, candelilla wax, carnauba wax, Japan wax, esparato grass wax, cork wax, guaruma wax, rice germ oil wax, sugar cane wax, berry wax, ouricury wax, montan wax, jojoba wax, shea butter, beeswax, shellac wax, spermaceti, lanolin (wool wax), uropygial grease, ceresin, ozokerite (earth wax), paraffin waxes and microcrystalline waxes are favorable.

Further advantageous fat and/or wax components are chemically modified waxes and synthetic waxes, such as, for example, those available under the trade names Syncrowax HRC (glyceryl tibehenate), Syncrowax HGLC (C₁₆₋₃₀-fatty acid triglyceride) and Syncrowax AW 1C (C₁₈₋₃₆-fatty acid) from CRODA GmbH, and also montan ester waxes, sasol waxes, hydrogenated jojoba waxes, synthetic or modified beeswaxes (e.g. dimethicone copolyol beeswax and/or C₃₀₋₅₀-alkyl beeswax), polyalkylene waxes, polyethylene glycol waxes, but also chemically modified fats, such as, for example, hydrogenated vegetable oils (for example hydrogenated castor oil and/or hydrogenated coconut fatty glycerides), triglycerides, such as, for example, trihydroxystearin, fatty acids, fatty acid esters and glycol esters, such as, for example, C₂₀₋₄₀-alkyl stearate, C₂₀₋₄₀-alkylhydroxystearoylstearate and/or glycol montanate. Also advantageous are certain organosilicon compounds, which have similar physical properties to the specified fat and/or wax components, such as, for example, stearoxytrimethylsilane, provided the conditions required in the main claim are observed.

According to the invention the fat and/or wax components can be present either individually or as a mixture.

Any desired mixtures of such oil and wax components are also to be used advantageously within the context of the present invention.

The oil phase is advantageously selected from the group 2-ethylhexyl isostearate, octyldodecanol, isotridecyl isononanoate, butylene glycol dicaprylate/dicaprate, 2-ethylhexyl cocoate, C12-15-alkyl benzoate, caprylic/capric acid triglyceride, dicapryl ether. Particularly advantageous mixtures are those of octyldodecanol, caprylic/capric acid triglyceride, dicaprylyl ether, dicaprylyl carbonate, cocoglycerides, or mixtures of C12-15-alkyl benzoate and 2-ethylhexyl isostearate, mixtures of C12-15-alkyl benzoate and butylene glycol dicaprylate/dicaprate, and mixtures of C12-15-alkyl benzoate, 2-ethylhexyl isostearate and isotridecyl isononanoate.

Particularly advantageous mixtures are those of octyldodecanol, caprylic/capric acid triglyceride, dicaprylyl ether, dicaprylyl carbonate, cocoglycerides, or mixtures of C12-15-alkyl benzoate and 2-ethylhexyl isostearate, mixtures of C12-15-alkyl benzoate and butylene glycol dicaprylate/dicaprate, and mixtures of C12-15-alkyl benzoate, 2-ethylhexyl isostearate and isotridecyl isononanoate.

Of the hydrocarbons, paraffin oil, cycloparaffin, squalane, squalene, hydrogenated polyisobutene and polydecene are to be used advantageously within the context of the present invention.

W/O emulsions according to the invention can advantageously be produced with the help of customary W/O emulsifiers, if desired with the aid of O/W emulsifiers or further coemulsifiers.

W/O emulsions according to the present invention can also comprise one or more emulsifiers selected from the group of the following substances which generally act as W/O emulsifiers:

lecithin, lanolin, microcrystalline wax (cera microcristallina) in a mixture with paraffin oil (paraffinum liquidum), ozokerite, hydrogenated castor oil, polyglyceryl-3 oleate, wool wax acid mixtures, wool wax alcohol mixtures, pentaerythrityl isostearate, polyglyceryl-3 diisostearate, beeswax (cera alba) and stearic acid, sodium dihydroxycetylphosphate in a mixture with isopropyl hydroxycetyl ether, methylglucose dioleate, methylglucose dioleate in the mixture with hydroxystearate and beeswax, mineral oil in a mixture with petrolatum and ozokerite and glyceryl oleate and lanolin alcohol, petrolatum in a mixture with ozokerite and hydrogenated castor oil and glyceryl isostearate and polyglyceryl-3 oleate, PEG-7 hydrogenated castor oil, ozokerite and hydrogenated castor oil, polyglyceryl-4 isostearate, polyglyceryl-4 isostearate in a mixture with cetyldimethicone copolyol and hexyl laurate, laurylmethicone copolyol, cetyldimethicone copolyol, acrylate/C₁₀₋₃₀-alkyl acrylate crosspolymer, polyoxamer 101, polyglyceryl-2 dipolyhydroxystearate, polyglyceryl-3 diisostearate, polyglyceryl-4 dipolyhydroxystearate, PEG-30 dipolyhydroxystearate, diisostearoyl polyglyceryl-3 diisostearate, polyglyceryl-2 dipolhydroxystearate, polyglyceryl-3 dipolyhydroxystearate, polyglyceryl-4 dipolyhydroxystearate, polyglyceryl-3 dioleate.

Furthermore, W/O emulsions corresponding to the present invention can comprise one or more coemulsifiers, in particular selected from the group of the following substances which generally act as O/W emulsifiers: glyceryl stearate in a mixture with ceteareth-20, ceteareth-25, ceteareth-6 in a mixture with stearyl alcohol, cetylstearyl alcohol in a mixture with PEG-40 castor oil and sodium cetylstearyl sulfate, triceteareth-4 phosphate, sodium cetylstearyl sulfate, lecithin trilaureth-4 phosphate, laureth-4 phosphate, stearic acid, propylene glycol stearate SE, PEG-25 hydrogenated castor oil, PEG-54 hydrogenated castor oil, PEG-6 caprylic/capric acid glycerides, glyceryl oleate in a mixture with propylene glycol, ceteth-2, ceteth-20, polysorbate 60, glyceryl stearate in a mixture with PEG-100 stearate, laureth-4, ceteareth-3, isostearyl glyceryl ether, cetylstearyl alcohol in a mixture with sodium cetylstearyl sulfate, laureth-23, steareth-2, glyceryl stearate in a mixture with PEG-30 stearate, PEG-40 stearate, glycol distearate, PEG-22 dodecyl glycol copolymer, polyglyceryl-2 PEG-4 stearate, ceteareth-20, methylglucose sesquistearate, steareth-10, PEG-20 stearate, steareth-2 in a mixture with PEG-8 distearate, steareth-21, steareth-20, isosteareth-20, PEG-45/dodecyl glycol copolymer, methoxy-PEG-22/dodecyl glycol copolymer, PEG-20 glyceryl stearate, PEG-20 glyceryl stearate, PEG-8 beeswax, polyglyceryl-2 laurate, isostearyl diglyceryl succinate, stearamidopropyl PG dimonium chloride phosphate, glyceryl stearate SE, ceteth-20, triethyl citrate, PEG-20 methylglucose sesquistearate, ceteareth-12, glyceryl stearate citrate, cetyl phosphate, triceteareth-4 phosphate, trilaureth-4 phosphate, polyglyceryl methylglucose distearate, potassium cetyl phosphate, isosteareth-10, polyglyceryl-2 sesquiisostearate, ceteth-10, oleth-20, isoceteth-20, glyceryl stearate in a mixture with ceteareth-20, ceteareth-12, cetylstearyl alcohol and cetyl palmitate, cetylstearyl alcohol in a mixture with PEG-20 stearate, PEG-30 stearate, PEG-40 stearate, PEG-100 stearate.

It may also be advantageous within the context of the present invention, particularly when the oil phase of the preparations consists at least partially of silicone oils, to use silicone emulsifiers. The silicone emulsifiers can advantageously be selected from the group of interface-active substances from the group of alkylmethicone copolyols and/or alkyldimethicone copolyols, in particular from the group of compounds characterized by the following chemical structure:

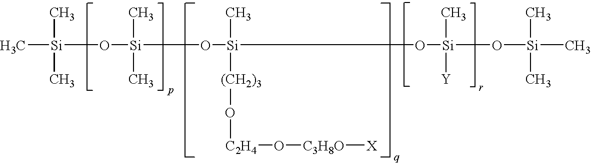

in which X and Y, independently of one another, are selected from the group H and the branched and unbranched alkyl groups, acyl groups and alkoxy groups having 1-24 carbon atoms, p is a number from 0-200, q is a number from 1-40, and r is a number from 1-100.

An example of silicone emulsifiers which are to be used particularly advantageously within the context of the present invention are dimethicone copolyols, which are sold by Th. Goldschmidt AG under the trade names ABIL® B 8842, ABIL® B 8843, ABIL® B 8847, ABIL® B 8851, ABIL® B8852, ABIL® B 8863, ABIL® 8873 and ABIL® 88183.

A further example of interface-active substances to be used particularly advantageously within the context of the present invention is cetyldimethicone copolyol, which is sold by Th. Goldschmidt AG under the trade name ABIL® EM 90.

A further example of interface-active substances to be. used particularly advantageously within the context of the present invention is the cyclomethicone dimethicone copolyol, which is sold by Th. Goldschmidt AG under the trade name ABIL® EM 97.

Furthermore, an emulsifier which has proven particularly advantageous is laurylmethicone copolyol, which is available under the trade name Dow Corning® 5200 Formulation Aid from Dow Corning Ltd.

The total amount of silicone emulsifiers used advantageously according to the invention in the cosmetic or dermatological preparations according to the invention is advantageously selected from the range from 0.1-10.0% by weight, preferably 0.5-5.0% by weight, based on the total weight of the preparations.

Emulsions according to the invention within the context of the present invention, e.g. in the form of a skin (protection) cream, a skin lotion, a cosmetic milk, for example in the form of a sun skin cream or a sun protection milk or aftersun product, comprise, for example, fats, oils, waxes and/or other fatty substances, and also water and one or more emulsifiers as are customarily used for such a type of formulation.

Just as emulsions of liquid and solid consistency according to the invention are used as cosmetic cleansing lotions or cleansing creams, the preparations according to the invention may also constitute sprayable cleansing preparations (“cleansing sprays”), which are used, for example, for removing make-up, as mild peeling lotion or as mild washing lotion—if appropriate also for blemished skin. Cleansing preparations of this type can advantageously be further used as so-called “rinse off preparations”, which are rinsed off from the skin following application.

In one embodiment of the invention, the compositions can also comprise liposomes, which may also be present in the form of forms known in the specialist field as microsomes, nanoliposomes or nanoparticles. Examples of such nanoparticles are described in EP-A 1 003 488, DE-U 29 923 848 or KR1020040015586 A.

The person skilled in the art is of course aware that sophisticated cosmetic compositions can in most cases not be produced without customary auxiliaries and additives. These include, for example, consistency regulators, film formers, stabilizers, fillers, preservatives, perfumes, substances for preventing foaming, dyes, pigments which have a coloring effect, thickeners, surface-active substances, emulsifiers, softening, moisturizing and/or humectant substances, antiinflammatory substances, additional active ingredients, such as vitamins or proteins, photoprotective agents, insect repellants, bactericides, virucides, water, salts, antimicrobially, proteolytically or keratolytically effective substances, medicaments or other customary constituents of a cosmetic or dermatological formulation, such as alcohols, polyols, polymers, foam stabilizers, organic solvents and also electrolytes.

Corresponding requirements apply mutatis mutandis to the formulation of medicinal preparations.

Medicinal topical compositions within the context of the present invention generally comprise one or more medicaments in an effective concentration. For the sake of simplicity, for a clearer distinction between cosmetic and medicinal use and corresponding products, reference is made to the legal provisions of the Federal Republic of Germany (e.g. Cosmetics Ordinance, Foods and Drugs Act).

Cosmetic or topical dermatological compositions for the purposes of the present invention can be used, according to their formulation, for example as suncream, skin (protection) cream, cleansing milk, sunscreen lotion, aftersun cream or lotion, nutrient cream, day or night cream, peeling cream etc.

By incorporating the baobab constituents and/or the extracts therefrom produced under gentle conditions, the effective substances present in the plant constituents develop their care and calming effect on the skin. In particular, before, during and after the action of UV rays on the skin, irritation of the skin due to chemical stress (radiation) and thermal stress (heat), which can lead to redness and burning of the skin, is alleviated, and regeneration of the skin is promoted. On account of the natural substances present in the plant constituents or extracts, e.g. antioxidants and flavonoids, the stress on the skin, e.g. during or after sunbathing, is significantly reduced. In cases of irritation or reddening of the skin as a result of mechanical stress as well, relatively rapid subsidence of the skin reaction (inflammation) occurs.

Moreover, the incorporation of, for example, fruit pulp, leaves or bark serves, on account of the high pectin content of these plant constituents, to promote the storage of moisture and also the entry of moisture into the skin. As a result, the skin is less stressed even under the action of heat, mechanical or chemical irritation, and exhibits higher elasticity and smoothness compared to skin treated with customary creams.

A further important field of application of the compositions of the present invention is the use as mask. In this connection, on the one hand the application of a composition according to the invention of suitable consistency as spreadable paste is possible, on the other hand the impregnation of fabrics or fiber mixtures (for example cotton wool, fleece, wipes, pads), which are then placed onto the face or other desired parts of the body. For the use as mask, preparations can in particular be used into which comminuted plant constituents are incorporated, where fruit pulp, bark, leaves and flowers are particularly suitable.

The incorporation of constituents of the baobab plant according to the invention into “ready-made masks” is also provided by the present invention. In particular, masks which are placed onto the desired body part immediately after wetting, e.g. collagen masks, can have an incorporated constituent from the baobab plant according to the invention. For example, such a ready-made mask can comprise comminuted plant material, preferably fruit pulp, bark constituents, flowers or leaves of the baobab plant, in a network of fibers, preferably collagen fibers.

One example of collagen masks into which plant constituents or extracts can be incorporated according to the invention are those which are described, for example, in DE-A 10 350 654. Such ready-made masks, which can serve as a basis for the present invention, are manufactured and sold, for example, by Dr. Suwelack Skin & Health Care AG, Germany.

For the incorporation of the comminuted plant constituents into masks of the described type, separation of the resulting comminuted constituents according to size is not necessary; instead, a broad particle size distribution may be present. Preferably, the plant constituents for all cosmetic or dermatological preparations of the present invention are comminuted to a particle size of less than 1000 μm, preferably to a particle size of less than 500 μm. The finer the particles, the lower the average particle size, the better the particles can be incorporated, for example, into creams or lotions. It is therefore preferred to keep the particle sizes of the incorporated particles in formulations that are to be applied thinly to the skin, such as creams or lotions, essentially below 300 μm, preferably essentially below 200 μm, particularly preferably below 100 μm. “Dust”, i.e. particles below 50 μm, preferably below 20 μm, can also be incorporated into formulations according to the invention.

The amount of comminuted plant constituents to be incorporated according to the invention into a “base composition” with respective desired properties (for example cream, lotion, mask, peeling mass etc.) can be up to 20% by weight depending on the base composition, but is preferably in the range between 0.01 and 10% by weight, in a preferred embodiment between 0.01 and 5% by weight. The range can particularly suitably be from 0.05 to 2% by weight, preferably from 0.05 to 1% by weight, depending in each case on the consistency and the formulation of the base composition.

When incorporating an extract, up to 50% by weight of such an extract can be incorporated into a formulation, e.g. a cream or lotion. Particularly if the extract has been produced with the help of aqueous solvents or oils, the extract can directly replace part of the aqueous phase or the oil phase of the base composition. Usually, however, the extracts are incorporated into the compositions in a range from 0.01 to 20% by weight, preferably 0.05 to 10% by weight, particularly preferably 0.05 to 5% by weight.

As already said, the amount of plant constituents to be incorporated and their particle size is, just like the amount of extracts to be incorporated, dependent on the base formulation used in each case. For a person skilled in the art in the field of cosmetics, it is easy to establish through a few experiments how much and which particle size of the plant constituents is to be incorporated into a formulation. For example, a larger amount of also coarser plant constituents can be incorporated into a spreadable paste which is applied to body parts in the form of a mask, than into a sun lotion that is to be applied thinly. Also when embodying the plant particles in “ready-made masks” (see above), coarser and more plant particles can be incorporated than into a care day cream since the consistency thereof is influenced, for example, on account of the pectins present in the plant particles.

It is also possible to use the compositions according to the invention as a base for pharmaceutical formulations into which pharmaceutically effective substances are incorporated. Such effective substances may be those specified below, or else any other substance which can be incorporated into the composition according to the invention without problems.

It is advantageous that the skincare product according to the invention is provided as galenic formulation. Galenic formulations are understood by the person skilled in the art as meaning hydrogels, hydrodispersion gel, fatty gels, ointments, emulsions, solutions, pastes, capsules, oils, sticks, suspensions, powders, microemulsions, nanoemulsions, water, shaking emulsions, setting compositions, lacquers or rinse-off formulations. All of these galenic formulations known to the person skilled in the art have proven to be an advantageous vehicle for the product according to the invention in order to make the advantageous properties useful for the user.

It is likewise possible to make use of the constituents according to the invention in the form of decorative cosmetics (make-up formulations).

One embodiment of the invention is cosmetic and dermatological preparations which are in the form of a UV-filtering cream or lotion, e.g. a sunscreen composition. Usually, these comprise at least one UVA filter substance and/or at least one UVB filter substance and/or at least one inorganic pigment. Moreover, it is nowadays customary to offer creams whose main purpose is not protection against sunlight but which nevertheless comprise a content of UV protection substances. Thus, for example, UV-A and/or UV-B filter substances are usually incorporated into day creams.

According to the present invention, besides at least one hitherto customary and known UV filter substance(s), at least one plant constituent or an extract from at least one constituent of a plant of the genus Adansonia is additionally incorporated into such compositions.

Investigations with extracts from constituents of the baobab plant revealed that such plant extracts not only produce a photoprotective effect themselves, but that in particular the incorporation of the extracts into compositions which comprised customary UV filters, brought about an improvement in the stability of these filters. In particular, UV filters that are not very stable under light, such as, for example, dibenzoylmethane derivatives, benzophenone derivatives or triazine derivatives, in particular trianilinotriazine derivatives, can be stabilized in the cosmetic preparations by extracts from the constitutes of the baobab plant. In this embodiment of the invention, i.e the composition which also comprises at least one of the known UV filters specified below, it is unimportant how the extract is produced. Differently to that described above, the extract used in such a composition can also be obtained by “hot extraction”, i.e. by an extraction method with a relatively long heating step. Nevertheless, a “cold extract” in accordance with the temperature limiting values stated above or the direct incorporation of at least one comminuted plant constituent is also preferred for this application.

The improvement in the stabilization of UV filters in sun creams through the addition of octocrylene is known, for example, from EP 815 835 B1.

Advantageous broadband filters or UV-B filter substances are, for example, bisresorcinyltriazine derivatives.with the following structure:

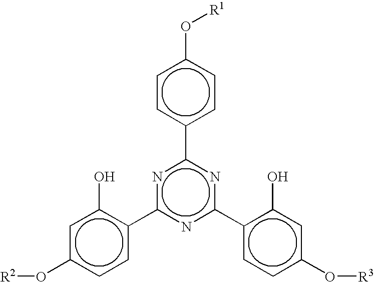

where R¹, R² and R³, independently of one another, are selected from the group of branched and unbranched alkyl groups having 1 to 10 carbon atoms or represent a single hydrogen atom. Particular preference is given to 2,4-bis{[4-(2-ethylhexyloxy)-2-hydroxy]phenyl}-6-(4-methoxyphenyl)-1,3,5-triazine (INCI: Aniso Triazine), which is available under the trade name Tinosorb® S from CIBA-Chemikalien GmbH.

Other UV filter substances which have the structural motive

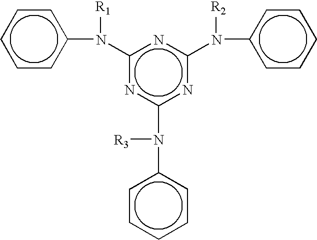

are also UV filter substances which may be present in formulations of the present invention, for example the s-triazine derivatives described in the European laid-open specification EP 570 838 A1, the chemical structure of which is given by the generic formula

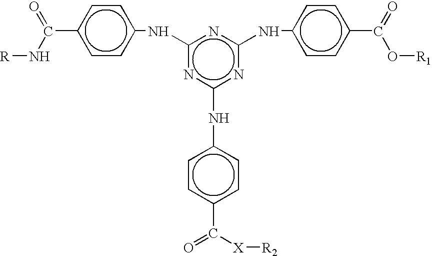

where

R is a branched or unbranched C₁-C₁₈-alkyl radical, a C₅-C₁₂-cycloalkyl radical, optionally substituted by one or more C₁-C₄-alkyl groups,

X is an oxygen atom or an NH group,

R₁ is a branched or unbranched C₁-C₁₈-alkyl radical, a C₅-C₁₂-cycloalkyl radical, optionally substituted by one or more C₁-C₄-alkyl groups, or is a hydrogen atom, an alkali metal atom, an ammonium group or a group of the formula

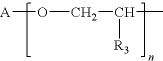

in which

A is a branched or unbranched C₁-C₁₈-alkyl radical, a C₅-C₁₂-cycloalkyl or aryl radical, optionally substituted by one or more C₁-C₄-alkyl groups,

R₃ is a hydrogen atom or a methyl group,

n is a number from 1 to 10,

R₂ is a branched or unbranched C₁-C₁₈-alkyl radical, a C₅-C₁₂-cycloalkyl radical, optionally substituted by one or more C₁-C₄-alkyl groups, if X is the NH group, and a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

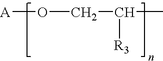

in which

A is a branched or unbranched C₁-C₁₈-alkyl radical, a C₅-C₁₂-cycloalkylalkyl or aryl radical, optionally substituted by one or more C₁-C₄-alkyl groups,

R₃ is a hydrogen atom or a methyl group,

n is a number from 1 to 10,

if X is an oxygen atom.

A further UV filter substance which can be incorporated according to the present invention is also an asymmetrically substituted s-triazine, which is also referred to below as bis-ethylhexylbutylamidotriazone (INCI: Dioctylbutamidotriazone) and is available under the trade name UVASORB HEB from Sigma 3V.

Within the context of the present invention it is also possible to incorporate a symmetrically substituted s-triazine, for example tris(2-ethylhexyl) 4,4′,4″-(1,3,5-triazine-2,4,6-triyltriimino)tris-benzoate, synonym: 2,4,6-tris[aniline(p-carbo-2′-ethyl-1′-hexyloxy)]-1,3,5-triazine (INCI: Octyl Triazone), which is sold by BASF Aktiengesellschaft under the trade name UVINUL® T 150.

European laid-open specification 775 698 also describes bisresorcinyltriazine derivatives that can be used, the chemical structure of which is given by the generic formula

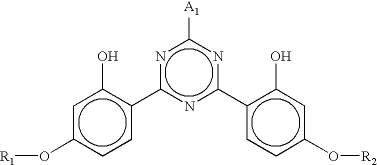

where R₁, R₂ and A represent very different organic radicals.

Within the context of the present invention it is also possible to incorporate 2,4-bis{[4-(3-sulfonato)-2-hydroxypropyloxy)-2-hydroxy]phenyl)-6-(4-methoxyphenyl)-1,3,5-triazine sodium salt, 2,4-bis{[4-3-(2-propyloxy)-2-hydroxypropyloxy)-2-hydroxy]phenyl)-6-(4-methoxyphenyl)-1,3,5-triazine, 2,4-bis{[4-(2-ethylhexyloxy)-2-hydroxy]phenyl}-6-[4-(2-methoxyethylcarboxyl)phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]phenyl}-6-(1-methylpyrrol-2-yl)-1,3,5-triazine, 2,4-bis{[4-tris(trimethylsiloxysilylpropyloxy)-2-hydroxy]phenyl}-6-(4-methoxyphenyl)-1,3,5-triazine, 2,4-bis{[4-(2″-methylpropenyloxy)-2-hydroxyphenyl}-6-(4-methoxyphenyl)-1,3,5-triazine and 2,4-bis{[4-(1′,1′,1′,3′,5′,5′,5′-heptamethylsiloxy-2″-methylpropyloxy)-2-hydroxy]phenyl}-6-(4-methoxyphenyl)-1,3,5-triazine.

The total amount of one or more triazine derivatives in the finished cosmetic or dermatological preparations, if present, is preferably selected from the range 0.01% by weight to 15% by weight, preferably from 0.1 to 10% by weight, in each case based on the total weight of the preparations.

Sulfonated, water-soluble UV filters can also be used within the context of the present invention: phenylene-1,4-bis(2-benzimidazyl)-3,3′-5,5′-tetrasulfonic acid, and its salts, particularly the corresponding sodium, potassium or triethanolammonium salts, in particular the phenylene-1,4-bis(2-benzimidazyl)-3,3′-5,5′-tetrasulfonic acid bis-sodium salt with the INCI name Bisimidazylate (CAS No.: 180898-37-7), which is available, for example, under the trade name Neo Heliopan AP from Haarmann & Reimer.

A further sulfonated UV filter that can be used within the context of the present invention is the salts of 2-phenylbenzimidazole-5-sulfonic acid, such as its sodium, potassium or its triethanolammonium salt, and the sulfonic acid itself with the INCI name Phenylbenzimidazole Sulfonic Acid (CAS No. 27503-81-7), which is available, for example, under the trade name Eusolex 232 from Merck or under Neo Heliopan Hydro from Haarmann & Reimer.

A further sulfonated UV filter is 3,3′-(1,4-phenylenedimethylene) bis(7,7-dimethyl-2-oxobicyclo[2.2.1]hept-1-ylmethanesulfonic acid, such as its sodium, potassium or its triethanolammonium salt, and the sulfonic acid itself with the INCI name Terephthalidene Dicamphor Sulfonic Acid (CAS No. 90457-82-2), which is available, for example, under the trade name Mexoryl SX from Chimex.

Further suitable water-soluble UV-B and/or broadband filter substances are, for example, sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

The total amount of one or more sulfonated UV filter substances in the finished cosmetic or dermatological preparations can be selected from the range 0.01% by weight to 20% by weight, preferably from 0.1 to 10% by weight, in each case based on the total weight of the preparations.

Also suitable are 1,4-di(2-oxo-10-sulfo-3-bornylidenemethyl)benzene and salts thereof (particularly the corresponding 10-sulfato compounds, in particular the corresponding sodium, potassium or triethanolammonium salt), which is also referred to as benzene-1,4-di(2-oxo-3-bornylidenemethyl-10-sulfonic acid).

The UV-B and/or broadband filters may be oil-soluble or water-soluble. Further oil-soluble UV-B and/or broadband filter substances are, for example:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethylamino)benzoate, amyl 4-(dimethylamino)benzoate;

derivatives of benzophenone, preferably 2-hydroxy-4-methoxybenzophenone, 2-hydroxy-4-methoxy-4′-methyl-benzophenone, 2,2′-dihydroxy-4-methoxybenzophenone

and UV filters bonded to polymers.

UV filter substances which are liquid at room temperature are, for example, homomenthyl salicylate (INCI: Homosalate), 2-ethylhexyl 2-cyano-3,3-diphenylacrylate (INCI: Octocrylene, for example available from BASF under the name Uvinul® N 539), 2-ethylhexyl 2-hydroxybenzoate (2-ethylhexyl salicylate, octyl salicylate, INCI: Octyl Salicylate, available for example from Haarmann & Reimer under the trade name Neo Heliopan OS) and esters of cinnamic acid, for example 2-ethylhexyl 4-methoxycinnamate (INCI: Octyl Methoxycinnamate, available for example from Hoffmann-La Roche under the trade name Parsol MCX) and isopentyl 4-methoxycinnamate (INCI: Isoamyl p-methoxycinnamate, available for example from Haarmann & Reimer under the trade name Neo Heliopan E 1000).

A further UV filter substance which is liquid at room temperature within the context of the present invention is a (3-(4-(2,2-bis-ethoxycarbonylvinyl)phenoxy)pro-penyl)methylsiloxane/dimethylsiloxane copolymer, which is available, for example, from Hoffmann-La Roche under the trade name Parsol SLX.

The total amount of one or more UV filter substances which are liquid at room temperature in the finished cosmetic or dermatological preparations can preferably be selected from the range 0.1% by weight to 30% by weight, preferably from 0.5 to 20% by weight, in each case based on the total weight of the preparations.

Preferred dibenzoylmethane derivatives that can be used as UVA filters within the context of the present invention are, in particular, 4-(tert-butyl)-4′-methoxydibenzoylmethane (CAS No. 70356-09-1), which is sold by Givaudan under the name Parsol 1789 and by Merck under the trade name Eusolex® 9020, and 4-isopropyldidenbzoylmethane (CAS No. 63250-25-9), which is sold by Merck under the name Eusolex 8020.

Benzotriazoles are characterized by the following structural formula:

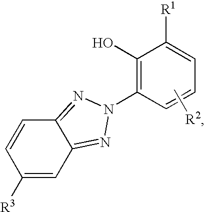

in which

R¹ and R², independently of one another, are linear or branched, saturated or unsaturated, substituted (e.g. substituted by a phenyl radical) or unsubstituted alkyl radicals having 1 to 18 carbon atoms and/or polymer radicals which themselves do not absorb UV rays (such as, for example, silicone radicals, acrylate radicals and the like), and

R³ is selected from the group H or alkyl radical having 1 to 18 carbon atoms.

A suitable benzotriazole within the context of the present invention is 2,2′-methylenebis(6-(2H-benzotriazol-2-yl)-4-(1,1,3,3-tetramethylbutyl)phenol), a broadband filter, which is available under the trade name Tinosorb® M from CIBA-Chemikalien GmbH.

A further suitable benzotriazole within the context of the present invention is also 2-(2H-benzotriazol-2-yl)-4-methyl-6-[2-methyl-3-[1,3,3,3-tetramethyl-1-[(trimethylsilyl)oxy]disiloxanyl]propyl)phenol (CAS No. 155633-54-8) with the INCI name Drometrizole Trisiloxane.

Further suitable benzotriazoles are [2,4′-dihydroxy-3-(2H-benzotriazol-2-yl)-5-(1,1,3,3-tetramethylbutyl)-2′-n-octoxy-5′-benzoyl]diphenylmethane, 2,2′-methylene-bis[6-(2H-benzotriazol-2-yl)-4-(methyl)phenol], 2,2′-methylenebis[6-(2H-benzotriazol-2-yl)-4-(1,1,3,3-tetramethylbutyl)phenol], 2-(2′-hydroxy-5′octyl-phenyl)benzotriazole, 2-(2′-hydroxy-3′,5′-di-t-amyl-phenyl)benzotriazole and 2-(2′-hydroxy-5′-methyl-phenyl)benzotriazole.

According to the invention, cosmetic or dermatological preparations can comprise 0.1 to 20% by weight, advantageously 0.5 to 15% by weight, very particularly preferably 0.5 to 10% by weight, of one or more benzotriazoles.

In a particularly preferred embodiment, the compositions according to the invention comprise, besides (one) plant constituent(s) or extract(s) from baobab of the abovementioned species, at least one UVA filter, preferably selected from the aforementioned dibenzoylmethane derivatives and/or benzophenone derivatives and/or at least one UVB filter, preferably selected from the specified tirazine derivatives, preferably trianilinotriazine derivatives, particularly preferably dioctylbutamidotriazone or octyltriazone, if appropriate octocrylene, but no cinnamic acid derivatives and/or no camphor derivatives as UV filter.

The constituents described below can be incorporated into the compositions according to the invention and can in each case individually or in combination develop an (further) advantageous effect. However, the constituents specified subsequently should not be understood as meaning obligatory constituents of a composition(s) according to the invention. Rather, any formulation suitable as cosmetic or dermatological formulation which comprises a constituent or extract according to the invention from a plant of the genus Adansonia, as specified above, should be regarded as falling under the invention.

Cosmetic and dermatological preparations according to the invention can comprise inorganic pigments based on metal oxides and/or other metal compounds that are insoluble or sparingly soluble in water, in particular the oxides of titanium (TiO₂), zinc (ZnO), iron (e.g. Fe₂O₃), zirconium (ZrO₂), silicon (SiO₂), manganese (e.g. Mno), aluminum (Al₂O₃), cerium (e.g. Ce₂O₃), mixed oxides of the corresponding metals, and mixtures of such oxides. These pigments are X-ray-amorphous or non-X-ray-amorphous. The pigments are particularly preferably based on TiO₂.

X-ray-amorphous oxide pigments are metal oxides or semimetal oxides which reveal no or no recognizable crystal structure in X-ray diffraction experiments. Such pigments are often obtainable by flame reaction, for example by reacting a metal or semimetal halide with hydrogen and air (or pure oxygen) in a flame.

In cosmetic, dermatological or pharmaceutical formulations, X-ray-amorphous oxide pigments are used as thickeners and thixotropic agents, flow auxiliaries, for emulsion and dispersion stabilization and as carrier substance (for example for increasing the volume of finely divided powders).

X-ray-amorphous oxide pigments which are known and often used in cosmetic or dermatological galenics are the silicon oxides of the Aerosil® type (CAS No.: 7631-86-9). Aerosils®, obtainable from DEGUSSA, are characterized by small particle size (e.g. between 5 and 40 nm), where the particles are to be regarded as spherical particles of very uniform dimension. Macroscopically, Aerosils® are recognizable as loose, white powders. Within the context of the present invention, X-ray-amorphous silicon dioxide pigments are particularly advantageous and, among these, especially those of the Aerosil® type are preferred.

Advantageous Aerosil® grades are, for example, Aerosil® OX50, Aerosil® 130, Aerosil® 150, Aerosil® 200, Aerosil® 300, Aerosil® 380, Aerosil® MOX 80, Aerosil® MOX 170, Aerosil® COK 84, Aerosil® R 202, Aerosil® R 805, Aerosil®R 812, Aerosil® R 972, Aerosil® R 974, Aerosil® R976.

The cosmetic or dermatological photoprotective preparations can comprise 0.1 to 20% by weight, advantageously 0.5 to 10% by weight, very particularly preferably 1 to 5% by weight, of X-ray-amorphous oxide pigments.

The non-X-ray-amorphous inorganic pigments are preferably present in hydrophobic form, i.e they have been surface-treated to repel water. This surface treatment may consist in providing the pigments with a thin hydrophobic layer by methods known per se.

One such method consists, for example, in producing the hydrophobic surface layer by a reaction according to n TiO₂+m(RO)₃Si—R′→n TiO₂ (surf.). Here, n and m are stoichiometric parameters to be used as desired, and R and R′ are the desired organic radicals. For example, hydrophobized pigments prepared. analogously to DE-A 33 14 742 are advantageous.

Organic surface coatings within the context of the present invention can consist of vegetable or animal aluminum stearate, vegetable or animal stearic acid, lauric acid, dimethylpolysiloxane (also: dimethicone), methylpolysiloxane (methicone), simethicone (a mixture of dimethylpolysiloxane with an average chain length.of from 200 to 350 dimethylsiloxane units and silica gel) or alginic acid. These organic surface coatings can occur on their own, in combination and/or in combination with inorganic coating materials.

Zinc oxide particles and predispersions of zinc oxide particles suitable according to the invention are available under the following trade names from the companies listed:

| Trade name | Coating | Manufacturer |
|---|---|---|
| Z-Cote HP1 | 2% dimethicone | BASF |
| Z-Cote | / | BASF |
| ZnO NDM | 5% dimethicone | H&R |
| MZ-505 S | 5% methicone | Tayca Corp. |

Suitable titanium dioxide particles and predispersions of titanium dioxide particles are available under the following trade names from the companies listed:

| Trade name | Coating | Manufacturer |
|---|---|---|
| MT-100TV | Aluminum | Tayca |
| | hydroxide/stearic acid | Corporation |
| MT-100Z | Aluminum | Tayco |
| | hydroxide/stearic acid | Corporation |
| Eusolex T-2000 | Alumina/simethicone | Merck KgaA |
| Titanium | Octyltrimethylsilane | Degussa |
| dioxide T805 | | |
| (Uvinul TiO2) | | |

Advantageous TiO₂ pigments are available, for example, under the trade name T 805, advantageous TiO/Fe₂O₃ mixed oxides are available under the trade name T 817 from Degussa.

The total amount of inorganic pigments, in particular hydrophobic inorganic micropigments, in the finished cosmetic or dermatological preparations is advantageously selected from the range from 0.1-0% by weight, preferably 0.1-10.0% by weight, in particular 0.5-6.0% by weight, based on the total weight of the preparations.

Besides the baobab constituents or extracts, the compositions according to the invention can also comprise extracts from other plants, in particular those extracts or oils which are known in cosmetics to be caring, protective or healing. Here, mention is to be made in particular of Aloe vera, jojoba oil, avocado oil, tea tree oil, soya germ extract, ginkgo extract and the like, without being limited to these.

Moreover, the cosmetic and dermatological preparations according to the invention can comprise cosmetic active ingredients, auxiliaries and/or additives, as are customarily used in such preparations, e.g. antioxidants, preservatives, bactericides, perfumes, substances for preventing foaming, dyes, pigments which have a coloring effect, thickeners, surface-active substances, emulsifiers, softening, moisturizing and/or humectant substances, fats, oils, waxes or other customary constituents of a cosmetic or dermatological formulation, such as alcohols, polyols, polymers, foam stabilizers, electrolytes, organic solvents or silicone derivatives.

Other ingredients known to be caring, such as, for example, taurine or ursolic acid, may also be present in the preparations according to the invention. On account of its sparing solubility, ursolic acid is not very accessible to skincare via customary cosmetic preparations. Ursolic acid therefore often occurs in combination with oleanolic acid, because oleanolic acid and ursolic acid are structurally related compounds which cannot technically be separated. The combination of ursolic acid and oleanolic acid is a constituent of customary commercial products and is therefore also part of the present invention. Preference is given to mixtures in the ranges from 60 to 95% by weight of ursolic acid and 5 to 40% by weight of oleanolic acid, based on the total mixture mass. Preference is given to a mixing ratio of from 70 to 90% by weight, in particular 80% by weight, of ursolic acid to 10 to 30% by weight, in particular 20% by weight, of oleanolic acid.

Preservatives approved in food technology, which are listed below with their E number, are to be used preferably according to the invention.

| | (regular grade) | HV | K | HS | S-728 |
|---|---|---|---|---|---|
| SiO₂ | 55.5 | 56.9 | 64.7 | 69.0 | 65.3 |
| MgO | 13.0 | 13.0 | 5.4 | 2.9 | 3.3 |
| Al₂O₃ | 8.9 | 10.3 | 14.8 | 14.7 | 17.0 |
| Fe₂O₃ | 1.0 | 0.8 | 1.5 | 1.8 | 0.7 |
| CaO | 2.0 | 2.0 | 1.1 | 1.3 | 1.3 |
| Na₂O | 2.1 | 2.8 | 2.2 | 2.2 | 3.8 |
| K₂O | 1.3 | 1.3 | 1.9 | 0.4 | 0.2 |
| Ashing | 11.1 | 12.6 | 7.6 | 5.5 | 7.5 |
| loss | | | | | |

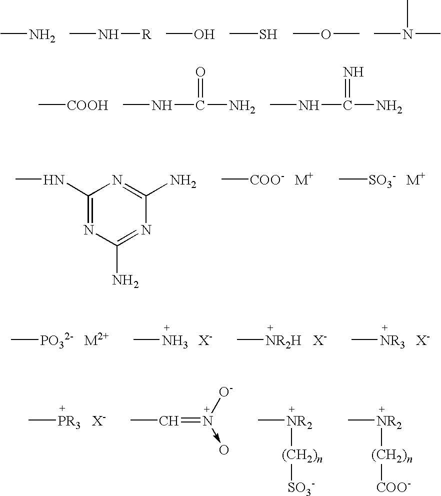

Also suitable according to the invention are preservatives or preservative aids customary in cosmetics: dibromodicyanobutane (2-bromo-2-bromommethylglutarodintrile), 3-iodo-2-propynyl butyl-carbamate, 2-bromo-2-nitropropane-1,3-diol, imidazolidinylurea, 5-chloro-2-methyl-4-isothiazolin-3-one, 2-chloroacetamide, benzylalkonium chloride, benzyl alcohol and/or formaldehyde donors.

Also suitable as preservatives are phenyl hydroxyalkyl ethers, in particular the compound known under the name phenoxyethanol, because of its bactericidal and fungicidal effects on a number of microorganisms.

Other antimicrobial agents are also likewise suitable for being incorporated into the preparations according to the invention. Advantageous substances are, for example, 2,4,4′-trichloro-2′-hydroxydiphenyl ether (Irgasan), 1,6-di(4-chlorophenylbiguanido)hexane (Chlorhexidin), 3,4,4′-trichlorocarbanilide, quaternary ammonium compounds, oil of cloves, mint oil, thyme oil, triethyl citrate, farnesol (3,7,11-trimethyl-2,6-10-dodecatrien-1-ol), and also the active ingredients or active ingredient combinations described in the patent laid-open specifications DE-37 40 186, DE-39 38 140, DE-42 04 321, DE-42 29 707, DE-43 09 372, DE-44 11 664, DE-195 41 967, DE-195 43 695, DE-195 43 696, DE-195 47 160, DE-196 02 108, DE-196 02 110, DE-196 02 111, DE-196 31 003, DE-196 31 004 and DE-196 34 019 and the patent specifications DE-42 29 737, DE-42 37 081, DE-43 24 219, DE-44 29 467, DE-44 23 410 and DE-195 16 705. Sodium hydrogen carbonate is also to be used advantageously.

Moreover, it may be advantageous to add further anti-irritative or anti-inflammatory active ingredients to the preparations within the context of the present invention, in particular batyl alcohol (α-octadecyl glyceryl ether), selachyl alcohol (α-9-octadecenyl glyceryl ether), chimyl alcohol (α-hexadecyl glyceryl ether), bisabolol and/or panthenol, although this is not necessary according to the invention.

It may likewise be advantageous to additionally add further customary antioxidants to the preparations within the context of the present invention. According to the invention, favorable antioxidants that can be used are all antioxidants that are customary or suitable for cosmetic and/or dermatological applications.

Suitable customary antioxidants can be selected from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, ψ-lycopene) and derivatives thereof, chlorogenic acid and derivatives thereof, aurothioglucose, propylthiouracil and other thiols (e.g thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to [mu]mol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, iminodisuccinate, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), and coniferyl benzoate of benzene resin, propyl gallate, ferulic acid, furfurylideneglucitol, carnosine, butylhydroxyltoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

The amount of the additional antioxidants (one or more compounds) in the preparations, if used, is preferably 0.001 to 30% by weight, particularly preferably 0.05-20% by weight, in particular 0.1-5% by weight, based on the total weight of the preparation.

If vitamin E and/or derivatives thereof are the antioxidant or antioxidants, it is advantageous to choose their respective concentrations from the range 0.001-10% by weight, based on the total weight of the formulation.

If vitamin A or vitamin A derivatives, or carotenes or derivatives thereof are the antioxidant or antioxidants, it is advantageous to choose their respective concentrations from the range from 0.001-10% by weight, based on the total weight of the formulation.

Preparations according to the present invention can also be used as a base for cosmetic or dermatological deodorants and antiperspirants. All active ingredients that are customary for deodorants and antiperspirants can advantageously be used, for example odor maskers such as the customary perfume constituents, odor. absorbers, for example the sheet silicates described in the patent laid-open specification DE-P 40 09 347, of these in particular montmorillonite, kaolinite, illite, beidellite, nontronite, saponite, hectorite, bentonite, smectite, also, for example, zinc salts of ricinoleic acid.

The amount of such active ingredients (one or more compounds) in the preparations according to the invention can preferably be 0.001 to 30% by weight, particularly preferably 0.05-20% by weight, in particular 1-10% by weight, based on the total weight of the preparation.

The water phase of the cosmetic preparations within the context of the present invention may also have gel character which, besides an effective content of substances used according to the invention and solvents customarily used therefor, preferably water, also further organic and/or inorganic thickeners.

The inorganic thickener or thickeners can, for example, be selected from the group of modified or unmodified, naturally occurring or synthetic sheet silicates.

Although it is entirely favorable to use pure components, it is also possible to incorporate mixtures of different modified and/or unmodified sheet silicates into the compositions according to the invention.

Within the context of this application, sheet silicates, which are also called phyllosilicates, are to be understood as meaning silicates and alumosilicates in which the silicate or aluminate units are joined together via three Si—O— or Al—O— bonds and form a corrugated sheet or layer structure. The fourth Si—O— or Al—O— valence is saturated by cations. Relatively weak electrostatic interactions exist between the individual layers, e.g. hydrogen bridge bonds. The layer structure, meanwhile, is largely defined by strong, covalent bonds.

The stoichiometry of the sheet silicates is

(Si₂O₅²⁻) for pure silicate structures and

(AlₘSi²⁻ₘO₅(^{2+m})⁻) for alumosilicates.

m is a number greater than zero and less than 2.

If pure silicates are not present, but alumosilicates are, the circumstance that each Si⁴⁺ group replaced by Al³⁺ requires a further singularly charged cation for charge neutralization is to be taken into consideration.

The charge balance is preferably evened out by H⁺, alkali metal ions or alkaline earth metal ions. Aluminum as counterion is also known and advantageous. In contrast to the alumosilicates, these compounds are called aluminum silicates. “Aluminum alumosilicates”, in which aluminum is present both in the silicate network, and also as counterion, are also known and, if appropriate, advantageous for the present invention.

Sheet silicates are well documented in the literature, e.g. in the “Lehrbuch der Anorganischen Chemie” [Textbook of Inorganic Chemistry], A. F. Hollemann, E. Wiberg and N. Wiberg, 91st-100th edition, Walter de Gruyter—Verlag 1985, passim, and “Lehrbuch der Anorganischen Chemie” [Textbook of Inorganic Chemistry], H. Remy, 12th edition, Akademische Verlagsgesellschaft, Leipzig 1965, passim. The layer structure of montmorillonite is given in Römpps Chemie-Lexikon, Franckh'sche Verlagshandlung W. Keller & Co., Stuttgart, 8th edition, 1985, p. 2668 et seq.

Examples of sheet silicates are:

| | |
|---|---|
| Montmorillonite | Na_{0.33}((Al_{1.67}Mg_{0.33})(OH)₂(Si₄O₁₀)) |
| often simplified: | Al₂O_{3.4}SiO₂•H₂O•nH₂O and |
| | Al₂[(OH)₂/Si₄O₁₀)•nH₂O |
| Kaolinite | Al₂(OH)₄(Si₂O₅) |
| Illite | (K,H₃O)_{y}(Mg₃)(OH)₂(Si_{4-y}Al_{y}O₁₀)) |
| and | (K,H₃O)_{y}(Al₂(OH)₂(Si_{4-y}Al_{y}O₁₀)) where |
| | y - 0.7-0.9 |
| Beidellite | (Ca,Na)_{0.3}(Al₂(OH)₂(Al_{0.5}Si_{3.5}O₁₀)) |
| Nontronite | Na_{0.33}(Fe₂)(OH)₂(Al_{0.33}Si_{3.67}O₁₀)) |
| Saponite | (Ca,Na)_{0.33}((Mg,Fe)₃(OH)₂(AlO_{0.33}Si_{3.67}O₁₀)) |
| Hectorite | Na_{0.33}((Mg,Li)₃(OH,F)₂(Si₄O₁₀)) |

Montmorillonite is the main mineral of the naturally occurring bentonites.

Within the context of the present invention, very advantageous inorganic gel formers are aluminum silicates, such as the montmorillonites (bentonites, hectorites and derivatives thereof, such as quaternium-18 bentonite, quaternium-18 hectorite, stearalkonium bentonites and stearalkonium hectorites), and also magnesium-aluminum silicates (Veegum® grades), and sodium-magnesium silicates (Laponite® grades).

Montmorillonites are clay minerals which are a type of dioctahedral smectites, and are masses which swell in water, but do not become plastic. The layer packets in the three-layer structure of the montmorillonites can swell as a result of reversible incorporation of water (in a 2-7-fold amount) and other substances, such as, for example, alcohols, glycols, pyridine, α-picoline, ammonium compounds, hydroxy-aluminosilicate ions etc.

The chemical formula given above is only approximate; since montmorillonites have a large capacity for ion exchange, Al can be replaced by Mg, Fe²⁺, Fe³⁺, Zn, Pb (e.g. from harmful substances in waste waters), Cr, and also Cu and others. The resulting negative charge of the octahedral layers is compensated by cations, in particular in Na⁺ (sodium montmorillonite) and Ca²⁺ (calcium montmorillonite is only swellable to a very small extent) in interlayer positions.

Synthetic magnesium silicates and/or bentonites that are advantageous within the context of the present invention are sold, for example, by Süd-Chemie under the trade name Optigel®.

An aluminum silicate that is advantageous within the context of the present invention is sold, for example, by R.T. Vanderbilt Comp., Inc., under the trade name Veegum®. The various Veegum® grades, which are all advantageous according to the invention, are characterized by the following compositions

| | (regular grade) | HV | K | HS | S-728 |
|---|---|---|---|---|---|
| SiO₂ | 55.5 | 56.9 | 64.7 | 69.0 | 65.3 |
| MgO | 13.0 | 13.0 | 5.4 | 2.9 | 3.3 |
| Al₂O₃ | 8.9 | 10.3 | 14.8 | 14.7 | 17.0 |
| Fe₂O₃ | 1.0 | 0.8 | 1.5 | 1.8 | 0.7 |
| CaO | 2.0 | 2.0 | 1.1 | 1.3 | 1.3 |
| Na₂O | 2.1 | 2.8 | 2.2 | 2.2 | 3.8 |
| K₂O | 1.3 | 1.3 | 1.9 | 0.4 | 0.2 |
| Ashing | 11.1 | 12.6 | 7.6 | 5.5 | 7.5 |
| loss | | | | | |

These products swell in water to form viscous gels which have an alkaline reaction. The organophilization of montmorillonite or bentonites (exchange of the interlayer cations for quaternary alkylammonium ions) produces products (bentones) which are preferably used for dispersion in organic solvents and oils, fats, ointments, paints, coatings and in detergents.

Bentone® is a trade name for various neutral and chemically inert gelling agents which are constructed from long-chain, organic ammonium salts and specific types of montmorillonite. Bentones swell in organic media and cause the latter to swell. The gels are stable in dilute acids and alkalis, although they partially lose their gelling properties upon prolonged contact with strong acids and alkalis. Because of their organophilic character, the bentones are only wettable by water with difficulty.

The following Bentone® grades are sold, for example, by Kronos Titan: Bentone® 27, an organically modified montmorillonite, Bentone® 34 (dimethyldioctylammonium bentonite), which is prepared in accordance with U.S. Pat. No. 2,531,427 and, because of its lipophilic groups, swells more readily in the lipophilic medium than in water, Bentone® 38, an organically modified montmorillonite, a cream-colored to white powder, Bentone® LT, a purified clay mineral, Bentone® Gel MIO, an organically modified montmorillonite which is supplied as a very fine suspension in mineral oil (SUS-71) (10% bentonite, 86.7% mineral oil and 3.3% wetting agent), Bentone® Gel IPM, an organically modified bentonite which is suspended in isopropyl myristate (10% bentonite, 86.7% isopropyl myristate, 3.3% wetting agent), Bentone® Gel CAO, an organically modified montmorillonite which is taken up in castor oil (10% bentonite, 86.7% castor oil and 3.3% wetting agent), Bentone® Gel Lantrol, an organically modified montmorillonite which, in paste form, is intended for further processing, in particular for the production, of cosmetic compositions; 10% bentonite, 64.9 lantrol (wool wax oil), 22.0 isopropyl myristate, 3.0 wetting agent and 0.1 propyl p-hydroxybenzoate, Bentone® Gel Lan I, a 10% strength Bentone® 27 paste in a mixture of wool wax USP and isopropyl palmitate, Bentone® Gel Lan II, a bentonite paste in pure liquid wool wax, Bentone® Gel NV, a 15% strength Bentone® 27 paste in dibutyl phthalate, Bentone® Gel OMS, a bentonite paste in Shellsol T., Bentone® Gel OMS 25, a bentonite paste in isoparaffinic hydrocarbons (Idopar® H), Bentone® Gel IPP, a bentonite paste in isopropyl palmitate.

“Hydrocolloid” is the technological abbreviation for the more correct term “hydrophilic colloid”. Hydrocolloids are macromolecules which have a largely linear structure and have intermolecular forces of interaction which permit secondary and primary valence bonds between the individual molecules and thus the formulation of a reticular structure. Some are water-soluble natural or synthetic polymers which, in aqueous systems, form gels or viscous solutions. They increase the viscosity of the water by either binding water molecules (hydration) or else by absorbing and encapsulating the water into their interwoven macromolecules, at the same time restricting the mobility of the water. Such water-soluble polymers represent a large group of chemically very different natural and synthetic polymers whose common feature is their solubility in water and/or aqueous media. A prerequisite for this is that these polymers have a number of hydrophilic groups sufficient for solubility in water and are not too greatly crosslinked. The hydrophilic groups can be nonionic, anionic or cationic in nature, for example as follows:

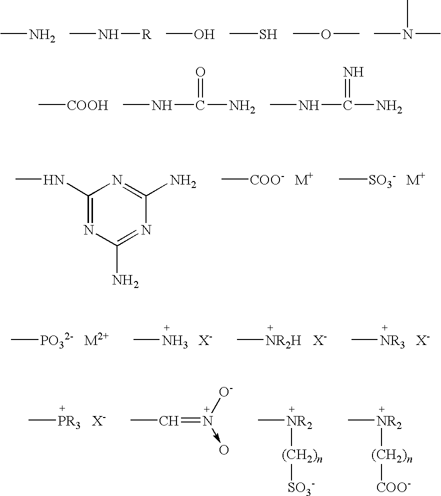

The group of the cosmetically and dermatologically relevant hydrocolloids can be divided. as follows into:

organic, natural compounds, such as, for example, agar agar, carrageen, tragacanth, gum Arabic, alginates, pectins, polyoses, guar flour, carob seed flour, starch, dextrins, gelatins, casein,

organic, modified natural substances, such as, for example, carboxymethylcellulose and other cellulose ethers, hydroxyethylcellulose and hydroxypropylcellulose and microcrystalline cellulose and the like,

organic, completely synthetic compounds, such as, for example, polyacrylic and polymethacrylic compounds, vinyl polymers, polycarboxylic acids, polyethers, polyimines, polyamides, polyurethanes,

inorganic compounds, such as, for example, polysilicic acids, clay minerals, such as montmorillonites, zeolites, silicas.

Microcrystalline cellulose is an advantageous hydrocolloid within the context of the present invention. It is available, for example, from “FMC Corporation Food and Pharmaceutical Products” under the trade name Avicel®. A particularly advantageous product within the context of the present invention is the grade Avicel® RC-591, which is a modified microcrystalline cellulose which is made up of 89% microcrystalline cellulose and 11% sodium carboxymethylcellulose. Further commercial products from this class of raw materials are Avicel® RC/CL, Avicel® CE-15, Avicel® 500.

Further hydrocolloids advantageous according to the invention are, for example, methylcelluloses, which is the term used for the methyl ethers of cellulose.

Of particular suitability within the context of the present invention are the cellulose mixed ethers, which are generally likewise referred to as methylcelluloses, which, besides a dominant content of methyl groups, additionally contain 2-hydroxyethyl, 2-hydroxypropyl or 2-hydroxybutyl groups. Particular preference is given to (hydroxypropyl)methylcelluloses, for example those available under the trade name Methocel® E4M from Dow Chemical Comp.

Also suitable according to the invention is sodium carboxymethylcellulose, the sodium salt of the glycolic acid ether of cellulose, for which R in structural formula I may be a hydrogen and/or CH₂—COONa.

Particular preference is given to the sodium carboxymethylcellulose referred to as cellulose gum and available under the trade name Natrosol Plus 330 CS from Aqualon.

Also preferred within the context of the present invention is xanthan (CAS No. 11138-66-2), also called xanthan gum, which is an anionic heteropolysaccharide which is generally formed by fermentation from corn sugar and is isolated as the potassium salt. It is produced by Xanthomonas campestris and a few other species under aerobic conditions and has a molecular weight of from 2×10⁶ to 24×10⁶. Xanthan is formed from a chain having β-1,4-bonded glucose (cellulose) with side chains. The structure of the subgroups consists of glucose, mannose, glucuronic acid, acetate and pyruvate. Xanthan is the name for the first microbial anionic heteropolysaccharide. It is produced by Xanthomonas campetris and a few other species under aerobic conditions and has a molecular weight of 2-15·10⁸. Xanthan is formed from a chain with β-1,4-bonded glucose (cellulose) with side chains. The structure of the subgroups consists of glucose, mannose, glucuronic acid, acetate and pyruvate. The number of pyruvate units determines the viscosity of the xanthan. Xanthan is produced in two-day batch cultures with a yield of 70-90%, based on carbohydrate used. Yields of 250 g/l are achieved here. After killing the culture, processing takes place by precipitation with e.g. 2-propanol. Xanthan is then dried and ground.

An advantageous gel former within the context of the present invention is also carrageen, a gel-forming extract with a similar structure to agar, of North Atlantic red algae which belong to the Florideae (Chondrus crispus and Gigartina stellata).

The term carrageen is frequently used for the dried algae product and carrageenan for the extract thereof. The carrageen precipitated from the hot-water extract is a colorless to sand-colored powder with a molecular weight range from 100 000 to 800 000 and a sulfate content of about 25%. Carrageen, which is very readily soluble in warm water, forms a thioxtropic gel upon cooling, even if the water content is 95-98%. The rigidity of the gel is brought about by the double helix structure of carrageen. In the case of carrageenan, three principle constituents are differentiated: the gel-forming κ fraction consists of D-galactose 4-sulfate and 3,6-anhydro-α-D-galactose, which has alternate glucoside bonds in the 1,3 and 1,4 position (by contrast, agar contains 3,6-anhydro-α-L-galactose). The nongelling [lambda]-fraction is composed of 1,3-glycosidically linked D-galactose-2-sulfate and 1,4-bonded D-galactose-2,6-disulfate radicals, and is readily soluble in cold water. [iota]-carrageenan, composed of D-galactose-4-sulfate in 1,3 bond and 3,6-anhydro-α-D-galactose 2-sulfate in 1,4 bond, is both water-soluble and also gel-forming. Further carrageen grades are likewise referred to with the Greek letters: α, β, γ, ν, ξ, π, ω, χ. The nature of cations which are present (K⁺, NH₄⁺, Na⁺, Mg²⁺, Ca²⁺) also influences the solubility of the carrageens.

The use of chitosan in cosmetic preparations is known per se. Chitosan is a partially deacylated chitin. This biopolymer has, inter alia, film-forming properties and is characterized by a silky skin feel. A disadvantage, however, is its considerable stickiness on the skin which arises in particular—temporarily—during use.

Chitosan is known for use, for example, in haircare. It is suitable, more so than the chitin on which it is based, as a thickener or stabilizer and improves the adhesion and water resistance of polymeric films. By way of representation of a large number of references in the prior art: H. P. Fiedler, “Lexikon der Hilfsstoffe für Pharmazie, Kosmetik und angrenzende Gebiete” [Lexikon of auxiliaries for pharmacy, cosmetics and related fields], 3rd edition 1989, Edition Cantor, Aulendorf, p. 293, keyword “Chitosan”.

Chitosan is characterized by the following structural formula:

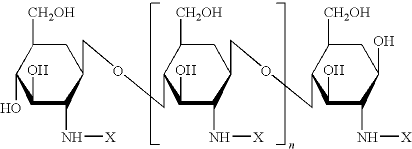

here, n assumes values up to about 10 000, X is either the acetyl radical or hydrogen. Chitosan is formed by deaceylation and partial depolymerization (hydrolysis) of chitin, which is characterized by the structural formula:

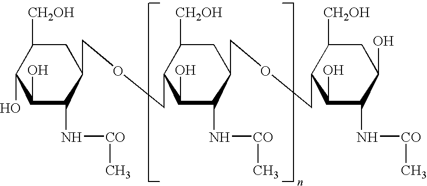

Chitin is an essential constituent of the ectoskeleton [oχιων=Greek: integument] of arthropods (e.g. insects, crabs, spiders) and is also found in protective tissues of other organisms (e.g. molluscs, algae, fungi).

In the region of about pH<6, chitosan is positively charged and also soluble in aqueous systems. It is not compatible with anionic raw materials. Consequently, for producing chitosan-containing oil-in-water emulsions, the use of nonionic emulsifiers is appropriate. These are known per se, for example from EP-A 776 657.

According to the invention, preference is given to chitosans with a degree of deacetylation of >25%, in particular >55 to 99% [determined by means of ¹H-NMR]).

It is advantageous to select chitosans with molecular weights between 10 000 and 1 000 000, in particular those with molecular weights between 100 000 and 1 000 000 [determined by means of gel permeation chromatography].

Polyacrylates are gelling agents that are likewise advantageous within the context of the present invention. Polyacrylates advantageous according to the invention are acrylate-alkyl acrylate copolymers, in particular those which are selected from the group of so-called carbomers or carbopols (Carbopol® is actually a registered trade mark of B.F. Goodrich Company). In particular, the acrylate-alkyl acrylate copolymers advantageous according to the invention are characterized by the following structure:

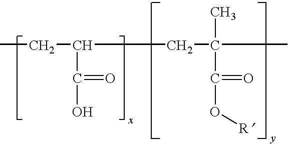

Here, R′ is a long-chain alkyl radical and x and y are numbers which symbolize the respective stoichiometric number of each of the comonomers.

According to the invention, particular preference is given to acrylate copolymers and/or acrylate-alkyl acrylate copolymers which are available under the trade names Carbopol® 1382, Carbopol® 981 and Carbopol® 5984 from B.F. Goodrich Company, preferably polyacrylates from the group of carbopols of grades 980, 981, 1382, 2984, 5984, and particularly preferably Cabopol Ultrez.

Also advantageous are copolymers of C10-30-alkyl acrylates and one or more monomers of acrylic acid, of methacrylic acid or esters thereof which are crosslinked with an allyl ether of sucrose or an allyl ether of pentaerythritol.

Compounds which bear the INCI name “acrylates/C10-30 alkyl acrylate crosspolymer” are advantageous. Those available under the trade names Pemulen TR1 and Pemulen TR2 from B.F. Goodrich Company are particularly advantageous.

Compounds which bear the INCI name ammonium acryloyldimethyltaurates/vinylpyrrolidone copolymers are advantageous.

According to the invention, the ammonium acryloyl-dimethyltaurates/vinylpyrrolidone copolymer(s) have the empirical formula [C₇H₁₆N₂SO₄]ₙ[C₆H₉NO]ₘ, corresponding to a statistical structure as follows

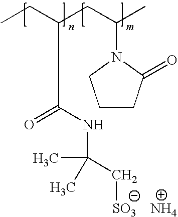

Preferred species within the context of the present invention are listed in the Chemical Abstracts under the registry numbers 58374-69-9, 13162-05-5 and 88-12-0 and are available under the trade name Aristoflex® AVC from Clariant GmbH.

Also advantageous are copolymers/crosspolymers comprising acryloyldimethyl taurates, such as, for example, Simugel® EG or Simugel® EG from Seppic S. A.

Within the context of the present concept, hydrocolloids from the group of anionic polymers are advantageously selected from the group of the carbomers as sodium, potassium, TEA and trisamino salts, sodium, potassium hyaluronate, microcrystalline cellulose+cellulose gum, veegum grades, hectorites, bentonites, laponites, alginates, methacrylates.

Within the context of the present concept, hydrocolloids from the group of nonionic polymers are advantageously selected from the group polyvinylpyrrolidone, hydroxypropylmethycellulose, polyvinyl alcohol, polyether-1, xanthan gum, hydroxyethylcellulose, cellulose derivatives, starch, starch derivative, guar gum, glyceryl methacrylate.

Within the context of the present concept, hydrocolloids from the group of cationic polymers are advantageously selected from the group chitosan, cationic starch derivatives, cationic cellulose derivatives, guar hydroxypropyltrimethylammonium chloride, sodium polystyrenesulfonate.

Suitable moisturizing and/or humectant agents (so-called moisturizers) within the context of the present concept are, for example, glycerol, lactic acid and/or lactates, in particular sodium lactate, butylene glycol, propylene glycol, biosaccaride gum-1, glycine soya, ethylhexyloxyglycerol, pyrrolidonecarboxylic acid and urea. It is also particularly advantageous to use polymeric moisturizers from the group of water-soluble and/or water-swellable and/or water-gellable polysaccharides. For example, hyaluronic acid, chitosan and/or a fucose-rich polysaccharide which is listed in the Chemical Abstracts under the registry number 178463-23-5 and is available, for example, under the name Fucogel® 1000 are particularly advantageous.

Within the context of the present invention, film formers are substances of varying composition which are characterized by the following property: if a film former is dissolved in water or other suitable solvents and the solution is then applied to the skin, then, following evaporation of the solvent, it forms a film which essentially has a protective function.

It is particularly advantageous to select the film formers from the group of polymers based on polyvinylpyrrolidone (PVP). Particular preference is given to copolymers of polyvinylpyrrolidone, for example the PVP hexadecene copolymer and the PVP eicosene copolymer, which are available under the trade names Antaron V216 and Antaron V220 from GAF Chemicals Cooperation.

It may be particularly advantageous to additionally stabilize W/O Pickering emulsions through the addition of copolymers of polyvinylpyrrolidone.

Further polymeric film formers, such as, for example, sodium polystyrenesulfonate, which is available under the trade name Flexan 130, and/or polyisobutene, available under the trade name Rewopal PIB1000, are likewise suitable.

The total amount of one or more film formers in the finished cosmetic or dermatological, for example W/O Pickering emulsions, is preferably chosen to be less than 10.0% by weight, further preferably between 1.0 and 7.0% by weight, based on the total weight of the preparations, and in O/W Pickering emulsions is preferably chosen to be less than 20.0% by weight, further preferably between 2.0 and 15.0% by weight, based on the total weight of the preparations.

Further hydrocolloids that can be used advantageously according to the invention are also

1. water-soluble or -dispersible anionic polyurethanes which are advantageously obtainable from

i) at least one compound which contains two or more active hydrogen atoms per molecule,

ii) at least one diol containing acid or salt groups and

iii) at least one diisocyanate.

Component i) is in particular a diol, aminoalcohol, diamine, polyesterol,. polyetherol with a number-average molecular weight of in each case up to 3000, or mixtures thereof, where up to 3 mol % of said compounds may be replaced by triols or triamines. Preference is given to diols and polyesterdiols. In particular, component (a) comprises at least 50% by weight, based on the total weight of component (a), of a polyesterdiol. Suitable polyesterdiols are all those which are customarily used for producing polyurethanes, in particular reaction products of phthalic acid and diethylene glycol, isophthalic acid and 1,4-butanediol, isophthalic acid/adipic acid and 1,6-hexanediol, and adipic acid and ethylene glycol or 5-NaSO₃-isophthalic acid, phthalic acid, adipic acid and 1,6-hexanediol.

Diols that can be used are, for example, ethylene glycol, propylene glycol, butylene glycol, neopentyl glycol, polyetherols, such as polyethylene glycols with molecular weights up to 3000, block copolymers of ethylene oxide and propylene oxide with number-average molecular weights of up to 3000 or block copolymers of ethylene oxide, propylene oxide and butylene oxide which contain the alkylene oxide units in randomly distributed manner or in the form of blocks. Preference is given to ethylene glycol, neopentyl glycol, di-, tri-, tetra-, penta- or hexaethylene glycol. Other diols that can be used are poly(α-hydroxycarboxylic acid) diols.

Suitable aminoalcohols are, for example, 2-aminoethanol, 2-(N-methylamino)ethanol, 3-aminopropanol or 4-aminobutanol.

Suitable diamines are, for example, ethylenediamine, propylenediamine, 1,4-diaminobutane and 1,6-diamino-hexane, and [alpha], [omega]-diamines which can be prepared by amination of polyalkylene oxides with ammonia.

Component ii) is, in particular, dimethylolpropanoic acid or compounds of the formulae

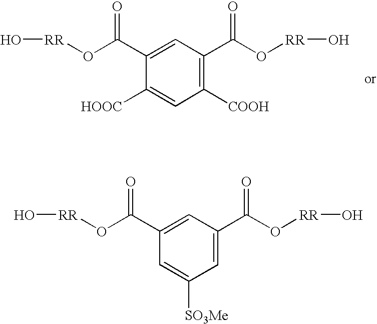

where RR is in each case a C₂-C₁₈-alkylene group and Me is Na or K.

Component iii) is, in particular, hexamethylene diisocyanate, isophorone diisocynate, methyldiphenyl isocyanate (MDI) and/or tolylene diisocyanate.

The polyurethanes are obtainable by reacting the compounds of groups i) and ii) under an inert-gas atmosphere in an inert solvent at temperatures of 70 to 130° C. with the compounds of group iii). This reaction can, if appropriate, be carried out in the presence of chain extenders in order to produce polyurethanes with relatively high molecular weights. As is customary in the production of polyurethanes, the components [(i)+(ii)]:iii) are advantageously used in the molar ratio from 0.8 to 1.1:1. The acid number of the polyurethanes is determined by the composition and the concentration of the compounds of component (ii) in the mixture of components (i)+(ii).

The polyurethanes have K values according to H. Fikentscher (determined in 0.1% strength by weight solutions in N-methylpyrrolidone at 25° C. and pH 7) of from 15 to 100, preferably 25 to 50.

The K value, also referred to as the intrinsic viscosity, is a parameter which is easy to determine via viscosity measurements of polymer solutions and is therefore frequently used in the industrial sector for characterizing polymers. For a certain type of polymer it is assumed, under standardized measuring conditions, to be solely dependent on the average molar mass of the investigated sample and is calculated using the relationship K value=1000 k in accordance with the Fikentscher equation

$$k = \frac{1.5 lg {η}_{r} - 1 \pm \sqrt{1 + ( \frac{2}{c} + 2 + 1.5 lg {η}_{r} ) - 1.5 lg {η}_{r}}}{150 + 300 c}$$

in which: εr=relative viscosity (dynamic viscosity of the solution/dynamic viscosity of the solvent) and c=mass concentration of polymer in the solution (in g/cm³)

The polyurethanes containing acid groups are, after neutralization (partial or complete), water-soluble or without the aid of emulsifiers. As a rule, the salts of the polyurethanes generally have better solubility or dispersibility in water than the unneutralized polyurethanes. Bases that can be used for the neutralization of the polyurethanes are alkali metal bases, such as sodium hydroxide solution, potassium hydroxide solution, soda, sodium hydrogen carbonate, potassium carbonate or potassium hydrogen carbonate and alkaline earth metal bases, such as calcium hydroxide, calcium oxide, magnesium hydroxide or magnesium carbonate, and also ammonia and amines. 2-Amino-2-methylpropanol, diethylaminopropylamine and triisopropanolamine have proven particularly useful for the neutralization of the polyurethanes containing acid groups. The neutralization of the polyurethanes containing acid groups can also be carried out using mixtures of two or more bases, e.g. mixtures of sodium hydroxide solution and triisopropanolamine. Depending on the intended use, neutralization may be partial, e.g. 20 to 40%, or complete, i.e. 100%.

These polymers and their production are described in more detail in DE-A-42 25 045, to which reference is hereby made in its entirety.

Further hydrocolloids that can advantageously be used according to the invention are

2. water-soluble or -dispersible, cationic polyurethanes and polyureas of

a) at least one diisocyanate, which may already have been reacted beforehand with one or more compounds which contain two or more active hydrogen atoms per molecule, and

b) at least one diol, primary or secondary aminoalcohol, primary or secondary diamine or primary or secondary triamine with one or more tertiary, quaternary or protonated tertiary amino nitrogen atoms.

Preferred diisocyanates are as given above under 1. iii). Compounds with two or more active hydrogen atoms are diols, aminoalcohols, diamines, polyesterols, polyamide diamines and polyetherols. Suitable compounds of this type are as given above under 1.

The production of the polyurethanes takes place as described under 1. Charged cationic groups can be produced in the polyureas from the tertiary amino nitrogen atoms present either by protonation, e.g. with carboxylic acids, such as lactic acid, or by quaternization, e.g. with alkylating agents, such as C₁- to C₄-alkyl halides or sulfates. Examples of such alkylating agents are ethyl chloride, ethyl bromide, methyl chloride, methyl bromide, dimethyl sulfate and diethyl sulfate.

These polymers and their production are described in more detail in DE-A-42 41 118, to which reference is hereby made in its entirety.

Further hydrocolloids that can be used advantageously according to the invention are

3. Linear polyurethanes with carboxylate groups of

i) a 2,2-hydroxymethyl-substituted carboxylic acid of the formula

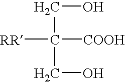

in which R,R′ is a hydrogen atom or a C₁-C₂₀-alkyl group, which is used in an amount which suffices for 0.35 to 2.25 milliequivalents of carboxyl groups to be present in the polyurethane per g of polyurethane,

ii) 10 to 90% by weight, based on the weight of the polyurethane, of one or more organic compounds with not more than two active hydrogen atoms and

iii) one or more organic diisocyanates.

The carboxyl groups present in the polyurethane are, finally, at least partially neutralized with a suitable base. These polymers and their production are described in EP-A-619 111, to which reference is hereby made in its entirety.

Further hydrocolloids to be used advantageously according to the invention are

4. carboxyl-containing polycondensation products of anhydrides of tri- or tetracarboxylic acids and diols, diamines or aminoalcohols (polyesters, polyamides or polyester amides). These polymers and their production are described in more detail in DE-A-42 24 761, to which reference is hereby made in its entirety.

Further hydrocolloids that can be used advantageously according to the invention are

5. polyacrylates and polymethacrylates, as are described in more detail in DE-A-43 14 305, 36 27 970 and 29 17 504. Reference is hereby made to these publications in their entirety.

The polymers that are possible according to the invention preferably have a K value of from 25 to 100, preferably 25 to 50. The polymers are generally present in the composition according to the invention in an amount in the range from 0.2 to 20% by weight, based on the total weight of the composition. The salt is generally used in an amount that is effective for improving the exchangeability of the polymers. In general, the salt is used in an amount of from 0.02 to 10% by weight, preferably 0.05 to 5% by weight and in particular 0.1 to 3% by weight, based on the total weight of the composition.

The total amount of one or more hydrocolloids in the finished cosmetic or dermatological preparations is advantageously chosen to be less than 5% by weight, preferably between 0.1 and 1.0% by weight, based on the total weight of the preparations.

In addition, it may be advantageous to add interface- or surface-active agents to preparations according to the invention, for example cationic emulsifiers such as, in particular quaternary surfactants.

Quaternary surfactants contain at least one N atom which is covalently bonded to 4 alkyl or aryl groups. This leads, irrespective of the pH, to a positive charge. Alkylbetaine, alkylamidopropylbetaine and alkylaminopropylhydroxysulfaine are advantageous. The cationic surfactants used according to the invention may also preferably be selected from the group of quaternary ammonium compounds, in particular benzyltrialkylammonium chlorides or bromides, such as, for example, benzyldimethylstearylammonium chloride, also alkyltrialkylammonium salts, for example cetyltrimethylammonium chloride or bromide, alkyldimethylhydroxyethylammonium chlorides or bromides, dialkyldimethylammonium chlorides or bromides, alkylamidoethyltrimethylammonium ether sulfates, alkylpyridinium salts, for example lauryl- or cetylpyrimidinium chloride, imidazoline derivatives and compounds with a cationic character, such as amine oxides, for example alkyldimethylamine oxides or alkylaminoethyldimethylamine oxides. In particular, cetyltrimethylammonium salts are to be used advantageously.

It is also possible to use cationic polymers (e.g. Jaguar® 162 [Hydroxypropyl Guar Hydroxpropyltrimonium Chloride] or modified magnesium aluminum silicates (e.g. quaternium-18-hectorite, which is available, for example, under the trade name Bentoneo 38 from Rheox, or stearalkonium hectorite, which is available, for example, under the trade name Softisan® Gel from Huls AG).

Preparations according to the invention can also comprise oil thickeners in order to improve the tactile properties of the emulsions and the stick consistency. Advantageous oil thickeners within the context of the present invention are, for example, further solids, such as, for example, hydrophobic silicon oxides of the Aerosil® grade, which are available from Degussa AG. Advantageous Aerosil® grades are, for example, Aerosil® OX50, Aerosil® 130, Aerosil® 150, Aerosil® 200, Aerosil® 300, Aerosil® 380, Aerosil® MOX 80, Aerosil® MOX 170, Aerosil® COK 84, Aerosil® 202, Aerosil® R 805, Aerosil® 812, Aerosil® R 972, Aerosil® R 974 and/or Aerosil® R976.

In addition, so-called metal soaps (i.e the salts of higher fatty acids with the exception of the alkali metal salts) are also suitable oil thickeners within the context of the present invention, such as, for example, aluminum stearate, zinc stearate and/or magnesium stearate.

It is likewise possible to add amphoteric and/or zwitterionic surfactants (e.g. cocoamidopropylbetaine) and moisturizers (e.g. betaine) to the preparations according to the invention. Examples of amphoteric surfactants to be used with preference are acyl/dialkylethylenediamine, for example sodium acylamphoacetate, disodium acylamphodipropionate, disodium alkylamphodiacetate, sodium acylampho-hydroxypropylsulfonate, disodium acylamphodiacetate and sodium acylamphopropionate, N-alkylamino acids, for example aminopropylalkylglutamide, alkylaminopropionic acid, sodium alkylimidodipropionate and lauroampho-carboxyglycinate.

The amount of the surface- and/or interface-active substances (one or more compounds) in the preparations according to the invention can preferably be 0.001 to 30% by weight, particularly preferably 0.05-20% by weight, in particular 1-10% by weight, based on the total weight of the preparation.

A surprising property of the preparations according to the invention is that they are very good vehicles for cosmetic or dermatological active ingredients into the skin, preferred active ingredients being, for example, the antioxidants present in the constituents of the baobab, which are able to protect the skin against oxidative stress.

The compositions according to the invention can also comprise pharmaceutical active ingredients (one or more compounds).

Of suitability are, for example, those from the group of lipophilic active ingredients, in particular from the following group:

acetylsalicylic acid, atropine, azulen, hydrocortisone and derivatives thereof, e.g. hydrocortisone-17 valerate, vitamins of the B and D series, very favorably vitamin B1, vitamin B5, vitamin B12, vitamin D1, but also bisabolol, unsaturated fatty acids, namely the essential fatty acids (often also called vitamin F), in particular [gamma]-linolenic acid, oleic acid, eicosapentaenoic acid, docosahexaenoic acid and derivatives thereof, chloramphenicol, caffeine, prostaglandins, dioic acid, allantoin, urea, thymol, camphor, extracts or other products of vegetable and animal origin, e.g. evening primrose oil, borage oil or blackcurrant seed oil, fish oils, cod liver oil and also ceramides and ceramide-like compounds, etc.

It is also possible to choose the active ingredients from the group of refatting substances, for example purcellin oil, Eucerit® and Neocerit®.

The active ingredient or ingredients can also be selected from the group of NO synthase inhibitors, particularly if the preparations according to the invention are to serve for the treatment and prophylaxis of the symptoms of intrinsic and/or extrinsic skin aging and also for the treatment and prophylaxis of the harmful effects of ultraviolet radiation on the skin.

A preferred NO synthase inhibitor is nitro arginine.

The active ingredient or ingredients can also be selected from the group which includes catechins and bile acid esters of catechins and aqueous or organic extracts from plants or parts of plants which have a content of catechins or bile acid esters of catechins, such as, for example, the leaves of the Theaceae plant family, in particular of the species Camellia sinensis (green tea.). Their typical ingredients (such as, for example, polyphenols or catechins, caffeine, vitamins, sugars, minerals, amino acids, lipids) are particularly advantageous.

Catechins are a group of compounds which are to be regarded as hydrogenated flavones or anthocyanidines and are derivatives of “catechin” (catechol, 3,3′,4′,5,7-flavanpentaol, 2-(3,4-dihydroxy-phenyl)chroman-3,5,7-triol). Epicatechin ((2R,3R)-3,3′,4′,5,7-flavanpentaol) is also an advantageous active ingredient within the context of the present invention.

Also advantageous are further plant extracts with a content of catechins, in particular extracts of green tea, such as, for example, extracts from leaves of the plants of the species Camellia spec., very particularly of the tea types Camellia sinensis, C. assamica, C. taliensis and C. irrawadiensis and hybrids of these with, for example, Camellia japonica.

Preferred active ingredients are also polyphenols and catechins from the group (−)-catechin, (+)-catechin, (−)-catechin gallate, (−)-gallocatechin gallate, (+)-epicatechin, (−)-epicatechin, (−)-epicatechin gallate, (−)-epigallocatechin, (−)-epigallocatechin gallate.

Flavone and its derivatives (also often collectively called “flavones”) are also advantageous active ingredients within the context of the present invention. They are characterized by the following basic structure (substitution positions are given):

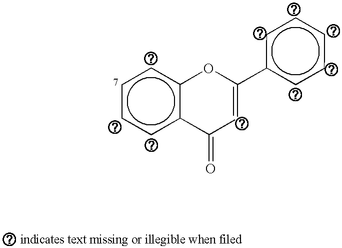

Various flavonoids, including the flavones, occur in the plant constituents used according to the invention here and can be extracted from these.

Some of the more important flavones which can also preferably (if appropriate additionally) be used in the preparations according to the invention are listed in table 2 below:

TABLE 2

| | OH substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones generally occur in glycosidated form.

According to the invention, the flavonoids are preferably selected from the group of substances of the generic structural formula

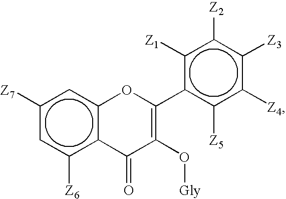

where Z₁ to Z₇, independently of one another, are selected from the group H, OH, alkoxy and hydroxyalkoxy, where the alkoxy and hydroxyalkoxy groups can be branched or unbranched and have 1 to 18 carbon atoms, and where Gly is selected from the group of mono- and oligoglycoside radicals.

According to the invention, the flavonoids can, however, also advantageously be selected from the group of substances of the generic structural formula

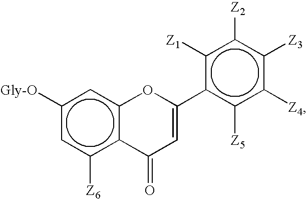

where Z₁ to Z₆, independently of one another, are selected from the group H, OH, alkoxy and hydroxyalkoxy, where the alkoxy and hydroxyalkoxy groups may be branched or unbranched and have 1 to 18 carbon atoms, and where Gly is selected from the group of mono- and oligoglycoside radicals.

Preferably, such structures can be selected from the group of substances of the generic structural formula

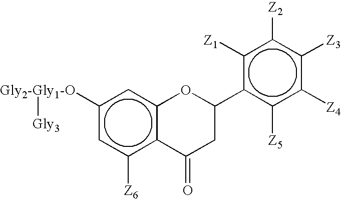

where Gly₁, Gly₂ and Gly₃, independently of one another, are monoglycoside radicals or. Gly₂ and Gly₃ may also, individually or together, represent saturations by hydrogen atoms.

Preferably, Gly₁, Gly₂ and Gly₃, independently of one another, are selected from the group of hexosyl radicals, in particular the rhamnosyl radicals and glucosyl radicals. However, other hexosyl radicals, for example allosyl, altrosyl, galactosyl, gulosyl, idosyl, mannosyl and talosyl, can also be used advantageously in some circumstances. It may also be advantageous according to the invention to use pentosyl radicals.

Z₁ to Z₅ are, independently of one another, advantageously selected from the group H, OH, methoxy, ethoxy and 2-hydroxyethoxy, and the flavone glycosides have the structure

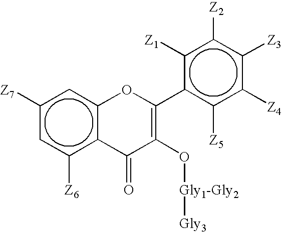

The flavone glycosides according to the invention are particularly advantageously from the group given by the following structure:

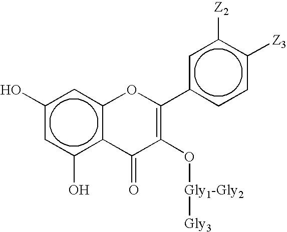

where Gly₁, Gly₂ and Gly₃, independently of one another, are monoglycoside radicals or. Gly₂ and Gly₃ can also, individually or together, represent saturations by hydrogen atoms.

Preferably, Gly₁, Gly₂ and Gly₃, independently of one another, are selected from the group of hexosyl radicals, in particular the rhamnosyl radicals and glucosyl radicals. However, other hexosyl radicals, for example allosy₁, altrosy₁, galactosy₁, gulosy₁, idosy₁, mannosyl and talosy₁, are also to be used advantageously in some circumstances. It may also be advantageous according to the invention to use pentosyl radicals.

Within the context of the present invention, it is particularly advantageous to select the flavone glycoside or glycosides from the group α-glucosylrutin, α-glucosylmyricetin, α-glucosylisoquercitrin, α-glu-cosylisoquercetin and α-glucosylquercitrin.

Also advantageous according to the invention are naringin (aurantin, naringenin-7-rhamnoglucoside), hesperidin (3′,5,7-trihydroxy-4′-methoxyflavanone-7-rutinoside, hesperidoside, hesperetin-7-O-rutinoside), rutin (3,3′,4′,5,7-pentahydroxyflavone-3-rutinoside, quercetin-3-rutinoside, sophorin, birutan, rutabion, taurutin, phytomelin, melin), troxerutin (3,5-di-hydroxy-3′,4′,7-tris(2-hydroxyethoxy)flavone-3-(6-O-(6-deoxy-α-L-mannopyranosyl)-β-D-glucopyranoside)), monoxerutin (3,3′,4′,5-tetrahydroxy-7-(2-hydroxy-ethoxy)flavone-3-(6-O-(6-deoxy-α-L-mannopyranosyl)-β-D-glucopyranoside)), dihydrorobinetin (3,3′,4′,5′,7-pentahydroxyflavanone), taxifolin (3,3′,4′,5,7-pentahydroxyflavanone), eriodictyol-7-glucoside (3′,4′,5,7-tetrahydroxyflavanone-7-glucoside), flavanomarein (3′,4′,7,8-tetrahydroxyflavanone-7-glucoside) and isoquercetin (3,3′,4′,5,7-pentahydroxyflavanone-3-(β-D-glucopyranoside).

It is also advantageous to select the active ingredient or active ingredients from the group of ubiquinones and plastoquinones.

Ubiquinones are characterized by the structural formula

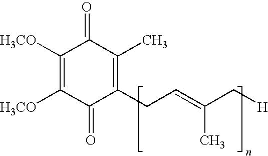

and are the most widespread and thus the most investigated bioquinones. Ubiquinones are referred to, depending on the number of isoprene units linked in the side chain, as Q-1, Q-2, Q-3 etc., or according to the number of carbon atoms, as U-5, U-10, U-15 etc. They preferably arise with certain chain lengths, e.g. in some microorganisms and yeasts where n=6. In most mammals including man, Q10 predominates.

Coenzyme Q10 is particularly advantageous and is characterized by the following structural formula:

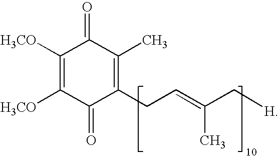

Plastoquinones have the general structural formula

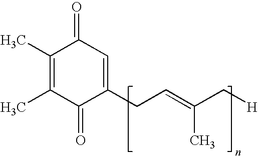

Plastoquinones differ in the number n of isoprene radicals and are referred to accordingly, e.g. PQ-9 (n=9). There are also other plastoquinones with varying substituents on the quinone ring.

Creatine and/or creatine derivatives are also preferred active ingredients within the context of the present invention. Creatine is characterized by the following structure:

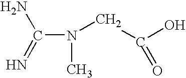

Preferred derivatives are creatine phosphate, and also creatine sulfate, creatine acetate, creatine ascorbate and the derivatives esterified at the carboxyl group with mono- or polyfunctional alcohols.

A further advantageous active ingredient is isoflavone 150 [3-hydroxy-4-(trimethylammonio)butyrobetaine. Acylisoflavone 150e, selected from the group of substances of the following general structural formula

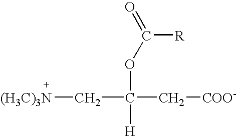

where R is selected from the group of branched and unbranched alkyl radicals having up to 10 carbon atoms are advantageous active ingredients within the context of the present invention. Preference is given to propionylcarnitine and in particular acetylcarnitine.

Both entantiomers (D and L form) are to be used advantageously within the context of the present invention. It may also be advantageous to use any desired enantiomer mixtures, for example a racemate of D and L form.

Further suitable active ingredients are sericoside, pyridoxol, vitamin K, biotin and aroma substances.

The list of specified active ingredients and active ingredient combinations which can be used in the preparations according to the invention is, of course, not intended to be limiting. The active ingredients can be used individually or in any combinations with one another, although none of the specified active ingredients—apart from the active ingredients according to the invention named in the claims—is categorically to be incorporated into the compositions according to the invention.

The amount of such active ingredients (one or more compounds) in the preparations according to the invention is preferably 0.001 to 10% by weight, particularly preferably 0.05-5% by weight, in particular 0.1-3% by weight, based on the total weight of the preparation.

In some cases, it may also be advantageous within the context of the present invention to incorporate dyes and/or colored pigments into the preparations according to the invention.

The dyes and colored pigments can be selected from the corresponding positive list of the Cosmetics Ordinance or the EC List of. Cosmetic Colorants. In most cases, they are identical to the dyes approved for foods. Advantageous colored pigments are, for example, titanium dioxide, mica, iron oxides (e.g. Fe₂O₃, Fe₃O₄, FeO(OH)) and/or tin oxide. Advantageous dyes are, for example, carmine, Prussian blue, chrome oxide green, ultramarine blue and/or manganese violet. It is particularly advantageous to select the dyes and/or colored pigments from the following list. The Colour Index Numbers (CIN) are taken from the Rowe Colour Index, 3rd edition, Society of Dyers and Colourists, Bradford, England, 1971.

| Chemical or other name | CIN | Color |
|---|---|---|
| Pigment Green | 10006 | green |
| Acid Green 1 | 10020 | green |
| 2,4-dinitrohydroxynaphthalene-7- | 10316 | yellow |
| sulfonic acid | | |
| Pigment Yellow 1 | 11680 | yellow |
| Pigment Yellow 3 | 11710 | yellow |
| Pigment Orange 1 | 11725 | orange |
| 2,4-dihydroxyazobenzene | 11920 | orange |
| Solvent Red 3 | 12010 | red |
| 1-(2′-chloro-4′-nitro-1′-phenylazo)- | 12085 | red |
| 2-hydroxynaphthalene | | |
| Pigment Red 3 | 12120 | red |
| Ceres red; Sudan red; Fat Red G | 12150 | red |
| Pigment Red 112 | 12370 | red |
| Pigment Red 7 | 12420 | red |
| Pigment Brown 1 | 12480 | brown |
| 4-(2′-methoxy-5′-sulfodiethylamido- | 12490 | red |
| 1′-phenylazo)-3-hydroxy-5″-chloro- | | |
| 2″,4″-dimethoxy-2-naphthanilide | | |
| Disperse Yellow 16 | 12700 | yellow |
| 1-(4-sulfo-1-phenylazo)-4- | 13015 | yellow |
| aminobenzene-5-sulfonic acid | | |
| 2,4-dihydroxyazobenzene-4′- | 14270 | orange |
| sulfonic acid | | |
| 2-(2,4-dimethylphenylazo-5-sulfo)-1- | 14700 | red |
| hydroxynaphthalene-4-sulfonic acid | | |
| 2-(4-sulfo-1-naphthylazo)-1-naphthol- | 14720 | red |
| 4-sulfonic acid | | |
| 2-(6-sulfo-2,4-xylylazo)-1-naphthol- | 14815 | red |
| 5-sulfonic acid | | |
| 1-(4′-sulfophenylazo)-2- | 15510 | orange |
| hydroxynaphthalene | | |
| 1-(2-sulfo-4-chloro-5-carboxy-1- | 15525 | red |
| phenylazo)-2-hydroxynaphthalene | | |
| 1-(3-methylphenylazo-4-sulfo)- | 15580 | red |
| 2-hydroxynaphthalene | | |
| 1-(4′,(8′)-sulfonaphthylazo)- | 15620 | red |
| 2-hydroxynaphthalene | | |
| 2-hydroxy-1,2′-azonaphthalene- | 15630 | red |
| 1′-sulfonic acid | | |
| 3-hydroxy-4-phenylazo-2- | 15800 | red |
| naphthylcarboxylic acid | | |
| 1-(2-sulfo-4-methyl-1-phenylazo)- | 15850 | red |
| 2-naphthylcarboxylic acid | | |
| 1-(2-sulfo-4-methyl-5-chloro-1- | 15865 | red |
| phenylazo)-2-hydroxynaphthalene- | | |
| 3-carboxylic acid | | |
| 1-(2-sulfo-1-naphthylazo)-2- | 15880 | red |
| hydroxynaphthalene-3-carboxylic acid | | |
| 1-(3-sulfo-1-phenylazo)-2-naphthol- | 15980 | orange |
| 6-sulfonic acid | | |
| 1-(4-sulfo-1-phenylazo)-2-naphthol- | 15985 | yellow |
| 6-sulfonic acid | | |
| Allura Red | 16035 | red |
| 1-(4-sulfo-1-naphthylazo)-2- | 16185 | red |
| naphthol-3,6-disulfonic acid | | |
| Acid Orange 10 | 16230 | orange |
| 1-(4-sulfo-1-naphthylazo)-2- | 16255 | red |
| naphthol-6,8-disulfonic acid | | |
| 1-(4-sulfo-1-naphthylazo)-2- | 16290 | red |
| naphthol-3,6,8-trisulfonic acid | | |
| 8-amino-2-phenylazo-1-naphthol- | 17200 | red |
| 3,6-disulfonic acid | | |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-sulfo-1-phenylazo)-1-(4- | 19140 | yellow |
| sulfophenyl)-5-hydroxypyrazolone- | | |
| 3-carboxylic acid | | |
| Pigment Yellow 16 | 20040 | yellow |
| 2,6-(4′-sulfo-2″,4″- | 20170 | orange |
| dimethyl)bisphenylazo)-1,3- | | |
| dihydroxybenzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-sulfo-1″-phenylazo)- | 27755 | black |
| 7′-sulfo-1′-naphthylazo]-1- | | |
| hydroxy-7-aminonaphthalene-3,6- | | |
| disulfonic acid | | |
| 4′-[(4″-sulfo-1″-phenylazo)- | 28440 | black |
| 7′-sulfo-1′-naphthylazo]-1- | | |
| hydroxy-8-acetylaminonaphthalene- | | |
| 3,5-disulfonic acid | | |
| Direct Orange 34, 39, 44, 46, 60 | 40215 | orange |
| Food Yellow | 40800 | orange |
| trans-β-Apo-8′-carotenaldehyde(C₃₀) | 40820 | orange |
| trans-Apo-8′-carotenic acid(C₃₀)- | 40825 | orange |
| ethyl ester | | |
| Canthaxanthin | 40850 | orange |
| Acid Blue 1 | 42045 | blue |
| 2,4-disulfo-5-hydroxy-4′-4″- | 42051 | blue |
| bis(diethylamino)triphenylcarbinol | | |
| 4-[(4-N-ethyl-p- | 42053 | green |
| sulfobenzylamino)phenyl(4-hydroxy-2- | | |
| sulfophenyl)(methylene)-1-(N-ethyl- | | |
| N-p-sulfobenzyl)-2,5-cyclohexadienimine] | | |
| Acid Blue 7 | 42080 | blue |
| (N-ethyl-p-sulfobenzylamino)phenyl(2- | 42090 | blue |
| sulfophenyl)methylene-(N-ethyl-N-p- | | |
| sulfobenzyl)Δ ^{2,5}-cyclohexadienimine | | |
| Acid Green 9 | 42100 | green |
| Diethyldisulfobenzyldi-4-amino-2- | 42170 | green |
| chloro-di-2-methyl-fuchsonimmonium | | |
| Basic Violet 14 | 42510 | violet |
| Basic Violet 2 | 42520 | violet |
| 2′-methyl-4′-(N-ethyl-N-m- | 42735 | blue |
| sulfobenzyl)amino-4″-(N-diethyl)amino- | | |
| 2-methyl-N-ethyl-N-m- | | |
| sulfobenzylfuchsonimmonium | | |
| 4′-(N-dimethyl)amino-4″-(N- | 44045 | blue |
| phenyl)aminonaphtho-N-dimethyl- | | |
| fuchsonimmonium | | |
| 2-hydroxy-3,6-disulfo-4,4′- | 44090 | green |
| bisdimethylaminonaphthofuchsonimmonium | | |
| Acid Red 52 | 45100 | red |
| 3-(2′-methylphenylamino)-6-(2′-methyl-4′- | 45190 | violet |
| sulfophenylamino)-9-(2″- | | |
| carboxyphenyl)xanthenium salt | | |
| Acid Red 50 | 45220 | red |
| Phenyl-2-oxyfluorone-2-carboxylic | 45350 | yellow |
| acid | | |
| 4,5-dibromofluorescein | 45370 | orange |
| 2,4,5,7-tetrabromofluorescein | 45380 | red |
| Solvent Dye | 45396 | orange |
| Acid Red 98 | 45405 | red |
| 3′,4′,5′,6′-tetrachloro-2,4,5,7- | 45410 | red |
| tetrabromofluorescein | | |
| 4,5-diiodofluorescein | 45425 | red |
| 2,4,5,7-tetraiodofluorescein | 45430 | red |
| Quinophthalone | 47000 | yellow |
| Quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-dioxyanthraquinone, calcium- | 58000 | red |
| aluminum complex | | |
| 3-oxypyrene-5,8,10-sulfonic acid | 59040 | green |
| 1-hydroxy-4-(N- | 60724 | violet |
| phenylaminoanthraquinone | | |
| 1-hydroxy-4-(4′- | 60725 | violet |
| methylphenylamino)anthraquinone | | |
| Acid Violet 23 | 60730 | violet |
| 1,4-di(4′- | 61565 | green |
| methylphenylamino)anthraquinone | | |
| 1,4-bis(o-sulfo-p- | 61570 | green |
| toluidino)anthraquinone | | |
| Acid Blue 80 | 61585 | blue |
| Acid Blue 62 | 62045 | blue |
| N,N′-dihydro-1,2,1′,2′- | 69800 | blue |
| anthraquinoneazine | | |
| Vat Blue 6; Pigment Blue 64 | 69825 | blue |
| Vat Orange 7 | 71105 | orange |
| Indigo | 73000 | blue |
| Indigo-disulfonic acid | 73015 | blue |
| 4,4′-dimethyl-6,6′-dichlorothioindigo | 73360 | red |
| 5,5′-dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanines | 74260 | green |
| Natural Yellow 6,19; Natural Red 1 | 75100 | yellow |
| Bixin, Nor-Bixin | 75120 | orange |
| Lycopene | 75125 | yellow |
| trans-alpha-, beta- and gamma-carotene | 75130 | orange |
| Keto- and/or hydroxyl derivatives | 75135 | yellow |
| of carotene | | |
| Guanine or pearlizing agent | 75170 | white |
| 1,7-Bis(4-hydroxy-3-methoxyphenyl)- | 75300 | yellow |
| 1,6-heptadiene-3,5-dione | | |
| Complex salt (Na, Al, Ca) of | 75470 | red |
| carminic acid | | |
| Chlorophyll a and b; copper | 75810 | green |
| compounds of chlorophylls and | | |
| chlorophyllins | | |
| Aluminum | 77000 | white |
| Hydrated alumina | 77002 | white |
| Hydrous aluminum silicates | 77004 | white |
| Ultramarine | 77007 | blue |
| Pigment Red 101 and 102 | 77015 | red |
| Barium sulfate | 77120 | white |
| Bismuth oxychloride and its | 77163 | white |
| mixtures with mica | | |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268:1 | black |
| Chromium oxide | 77288 | green |
| Chromium oxide, hydrous | 77289 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxide | 77491 | red |
| Iron oxide hydrate | 77492 | yellow |
| Iron oxide | 77499 | black |
| Mixtures of iron (II) and | 77510 | blue |
| iron(III)hexacyanoferrate | | |
| Pigment White 18 | 77713 | white |
| Manganese ammonium diphosphate | 77742 | violet |
| Manganese phosphate; Mn₃ (PO₄)₂•7H2O | 77745 | red |
| Silver | 77820 | white |
| Titanium dioxide and its mixtures | 77891 | white |
| with mica | | |
| Zinc oxide | 77947 | white |
| 6,7-dimethyl-9-(1′-D-ribityl)isoalloxazine, | | yellow |
| lactoflavine | | |
| Sugar coloring | | brown |
| Capsanthin, capsorubin | | orange |
| Betanin | | red |
| Benzopyrylium salts, anthocyans | | red |
| Aluminum, zinc, magnesium and | | white |
| calcium stearate | | |
| Bromothymol blue | | blue |
| Bromocresol green | | green |
| Acid Red 195 | | red |

It may also be favorable to select one or more substances from the following group as dye: 2,4-dihydroxyazobenzene, 1-(2′-chloro-4′-nitro-1′phenylazo)-2-hydroxynaphthalene, Ceres red, 2-(4-sulfo-1-naphthylazo)-1-naphthol-4-sulfonic acid, calcium salt of 2-hydroxy-1,2′-azonaphthalene-1′-sulfonic acid, calcium and barium salts of 1-(2-sulfo-4-methyl-1-phenylazo)-2-naphthylcarboxylic acid, calcium salt of 1-(2-sulfo-1-naphthylazo)-2-hydroxynaphthalene-3-carboxylic acid, aluminum salt of 1-(4-sulfo-1-phenylazo)-2-naphthol-6-sulfonic acid, aluminum salt of 1-(4-sulfo-1-naphthylazo)-2-naphthol-3,6-disulfonic acid, 1-(4-sulfo-1-naphthylazo)-2-naphthol-6,8-disulfonic acid, aluminum salt of 4-(4-sulfo-1-phenylazo)-1-(4-sulfophenyl)-5-hydroxypyrazolone-3-carboxylic acid, aluminum and zirconium salts of 4,5-dibromofluorescein, aluminum and zirconium salts of 2,4,5,7-tetrabromofluorescein, 3′,4′,5′6′-tetrachloro-2,4,5,7-tetrabromofluorescein and its aluminum salt, aluminum salt of 2,4,5,7-tetraiodofluorescein, aluminum salt of quinophthalonedisulfonic acid, aluminum salt of indigodisulfonic acid, red and black iron oxide (CIN: 77 491 (red) and 77 499 (black)), iron oxide hydrate (CIN: 77 492), manganese ammonium diphosphate and titanium dioxide.

Also of suitability are oil-soluble natural dyes, such as, for example, paprika extracts, β-carotene or cochineal.

Also advantageous for the present invention are gel creams with a content of pearlescent pigments. Preference is given in particular to the types of pearlescent pigments listed below:

natural pearlescent pigments, such as, for example,

“pearl essence” (guanine/hypoxanthin mixed crystals from fish scales) and

“mother of pearl” (ground mussel shells) monocrystalline pearlescent pigments, such as, for example, bismuth oxychloride (BiOCl) layer substrate pigments: e.g. mica/metal oxide

Bases for pearlescent pigments are, for example, pulverulent pigments or castor oil dispersions of bismuth oxychloride and/or titanium dioxide, and also bismuth oxychloride and/or titanium dioxide on mica. In particular, the color luster pigment listed under CIN 77163, for example, is advantageous.

Also of suitability are, for example, the following types of pearlescent pigment based on mica/metal oxide:

| Group | Coating/layer thickness | Color |
|---|---|---|
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Color luster pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Prussian blue | deep blue |
| | TiO₂/carmine | red |

Particular preference is given, for example, to the pearlescent pigments available from Merck under the trade names Timiron, Colorona and Dichrona.

The list of specified pearlescent pigments is not of course intended to be limiting. Pearlescent pigments that are advantageous within the context of the present invention are obtainable in numerous ways known per se.

For example, other substrates apart from mica can be coated with further metal oxides, such as, for example, silica and the like. SiO₂ particles coated with, for example, TiO₂ and Fe₂O₃ (“ronaspheres”), which are sold by Merck and are particularly suitable for the optical reduction of fine lines, are advantageous.

Moreover, it may be advantageous to dispense entirely with a substrate such as mica. Particular preference is given to pearlescent pigments produced using SiO₂. Such pigments, which may also additionally have gonichromatic effects, are available, for example, under the trade name Sicopeal Fantastico from BASF.

Furthermore, pigments from Engelhard/Mearl based on calcium sodium borosilicate which have been coated with titanium dioxide can be used. These are available under the name Reflecks. In addition to the color, as a result of their particle size of from 40-180 [mu]m, they have a glitter effect.

Of particular suitability are furthermore also effect pigments which are available under the trade name Metasomes Standard/Glitter in various colors (yellow, red, green, blue) from Flora Tech. The glitter particles are present here in mixtures with various auxiliaries and dyes (such as, for example, the dyes with the Colour Index (CI) numbers 19140, 77007, 77289, 77491).

The dyes and pigments can be present individually or in a mixture, and can be mutually coated with one another, differing coating thicknesses generally giving rise to different color effects. The total amount of dyes and color-imparting pigments is advantageously selected from the range from, for example, 0.1% by weight to 30% by weight, preferably from 0.5 to 15% by weight, in particular from 1.0 to 10% by weight, in each case based on the total weight of the preparations.

The preparations according to the invention are produced under the conditions familiar to the person skilled in the art. As a rule, the constituents of the oil phase or of the water phase are combined separately and heated, and then combined with one another with stirring and, preferably with homogenization, very particularly preferably with stirring with moderate to high input of energy, e.g. using a toothed-wheel dispersing machine at a rotational speed up to at most 10 000 rpm, preferably from 2500 to 7700 rpm.

### EXAMPLE 1

### Skin Cream Preparation

| Ingredients | | INCI name | %/100 g |
|---|---|---|---|
| 1 | Water | Aqua | 59.6 |
| 2 | RokonsalBS | Sodium benzoate/potassium benzoate | 1 |
| 3 | Glycerol | | 55 |
| 4 | Sorbitol | | |
| 5 | Keltrol TF | Xanthan gum | 0.2 |

Initially introduce positions 1-4, thoroughly homogenize in position 5,
heat to 75° C., cool

| | | | |
|---|---|---|---|
| 6 | Brij 72 | Steareth-2 | 3.5 |
| 7 | Brij 721 | Steareth-21 | 1.5 |
| 8 | Lanette O | Cetylstearyl alcohol | 3 |
| 9 | Baobab fruit pulp | Comminuted plant constituent | 10 |
| 10 | CetiolV | Decyl oleate | 5 |
| 11 | Prisorine 2039 | Isostearyl isostearate | 3 |
| 12 | Softisan 100 | Hydrogenated coconut glycerides | 3 |

Add positions 6-12, homogenize

| | | | |
|---|---|---|---|
| 13 | Perfume oil | Perfume oil | 0.2 |
| | | Add position 13 at 40° C. | |

1. A cosmetic or dermatological composition comprising an additive from at least one constituent of a plant of the genus Adansonia, in particular of the species Adansonia digitata (baobab plant), A. grandidieri, A. za, or A. gibbosa, wherein this additive is at least one fresh or dried plant constituent in comminuted form, and/or one extract from at least one plant constituent selected from the group consisting of fruit pulp, flowers and roots, or a combination thereof and wherein this additive is not obtained by hot extraction.

2. A cosmetic or dermatological composition comprising a UV protective filter substance and a fresh or dried plant constituent in comminuted form, and/or an extract selected from the group consisting of fruit pulps, barks, flowers, seeds and roots, or combinations thereof from a plant of the genus Adansonia, in particular of the species Adansonia digitata (baobab plant), A. grandidieri, A. za, or A. gibbosa.

3. The cosmetic or dermatological composition as claimed in claim 1 or 2, characterized in that the extract has been produced entirely under temperature conditions below at most at 65° C.

4. The cosmetic or dermatological composition as claimed in claims 1 or 2, wherein the plant constituent(s) are incorporated in comminuted form with a particle size below 1000 μM.

5. The cosmetic or dermatological composition as claimed in claims 1 or 2, wherein the extract is an aqueous extract, an extract with an oil or with an organic solvent.

6. The cosmetic dermatological composition as claimed in claims 1 or 2, wherein the incorporated fresh or dried plant constituent(s) is/are selected from the group consisting of fruit pulps, leaves, barks, flowers, seeds and roots, or combinations thereof.

7.-13. (canceled)

14. The cosmetic or dermatological composition as claimed in claim 1, wherein the composition comprises a fresh or dried plant constituent in comminuted form, as well as an extract from at least one plant constituent selected from the group consisting of fruit pulps, leaves, barks, flowers, seeds and roots, or combinations thereof.

15. The cosmetic or dermatological composition as claimed in claim 1, wherein the composition comprises 0.01 to 20% by weight, based on the total weight of the composition, of at least one comminuted plant constituent, and/or 0.01 to 50% by weight of an extract therefrom.

16. The cosmetic or dermatological composition as claimed in claim 1, wherein the composition is a sun cream, skin protection cream, cleansing milk, sunscreen lotion, aftersun cream or lotion, nutrient cream, day or night cream, peeling cream, peeling lotion or milk, ointment, a spreadable paste for use as a mask, ready-made masks based on fibrous fabric or nets, pads, or decorative cosmetics.

17. A method for producing an extract from at least one constituent selected from the group consisting of fruit pulps, flowers and roots of a plant of the genus Adansonia, in particular of the species Adansonia digitata (baobab plant), A. grandidieri, A. za or A. gibbosa, characterized in that the plant constituents are not subjected to a temperature above 65° C. during the production method until the extract is obtained.

18. The method as claimed in claim 17, wherein the extract is an aqueous extract, an extract based on oil or an extract based on an organic solvent.

19. The method as claimed in claim 17 or 18, wherein the extract is produced from at least one of the constituents selected from the group consisting of fruit pulps, leaves, barks, flowers, roots and seeds.
